(19) United States
(12) Patent Application Publication
(10) Pub. No.: US 2010/0323404 A1
(43) Pub. Date: Dec. 23, 2010

(54) METHOD FOR RECOMBINING DNA SEQUENCES AND COMPOSITIONS RELATED THERETO

(76) Inventor: Richard Lathrop, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/526,299

(22) PCT Filed: Feb. 8, 2008
(86) PCT No.: PCT/US08/53507

§ 371 Date: Aug. 7, 2009

Related U.S. Application Data
(60) Provisional application No. 60/889,251, filed on Feb. 9, 2007.

(51) Int. Cl. C12P 19/34; C40B 40/06; C07H 21/04

(52) U.S. Cl. 435/91.2; 506/16; 536/24.33

(57) ABSTRACT

Provided herein are methods for manipulating nucleotide sequences that permit greater control of sequence recombination compared to traditional methods, and related compositions. In one such method, a set of oligonucleotides and at least one primer are provided, where the primer has a first region uniquely complementary to a sequence of a first oligonucleotide of the set and a second region uniquely complementary to a second oligonucleotide of the set, combining the primer with an oligonucleotide comprising the first region of said first polynucleotide and an oligonucleotide comprising the second region of said second polynucleotide, and PCR amplifying to create a chimeric polynucleotide having some sequence from the first oligonucleotide and some sequence from the second oligonucleotide.

### CROSS-REFERENCE TO RELATED APPLICATIONS

This application claims priority to U.S. Provisional Application No. 60/889,251, filed Feb. 9, 2008, the entirety of which is incorporate by reference herein, where permitted.

### BACKGROUND OF THE INVENTION

1. Field of the Invention

Embodiments of the present invention relate to methods of recombining oligonucleotide sequences to develop new polynucleotide sequences and compositions comprising such sequences.

2. Description of the Related Art

A gene consists of a sequence of nucleotides that code for amino acids of a protein. Proteins are macromolecules that control an array of biological functions and therefore relate to an array of medical and industrial applications. By manipulating the nucleic acid sequence of a gene, the properties of the expressed protein are thereby affected.

Mutated genes are heavily used by a variety of fields. Biomedical researchers learn about and modify protein structure and function using mutated genes. The phenotypic effect of mutant genes that produce loss or gain of function can provide crucial clues about protein sequence-structure-function relationships. RNA splicing variants are a major source of protein diversity in higher eukaryotes. Frequently, an X-ray crystallographer must introduce mutations into a protein to obtain protein crystals for structure determination. Pharmacogenomics is an area of growing importance which is founded on the importance of understanding human DNA mutations and their effect on pharmaceuticals and biologics. Efforts to understand and control genetic diseases such as cancer would be enhanced by the ability to easily construct and study all of the important mutants in central pathways. Viral pathogens such as flu and HIV owe much of their impact to their ability to mutate more rapidly than natural defenses can follow. Mutations around a parental gene of interest are the main route to improved protein expression and function in biopharmaceuticals and industrial enzymes.

In some instances, specific known mutations are desired. One method of producing a mutated gene begins by dividing a gene into oligonucleotides, such that each oligonucleotide contains an extension that partially overlaps and is complementary to the adjacent oligonucleotide(s). These oligonucleotides are combined and allowed to anneal. Primer extension and Polymerase Chain Reaction (PCR) with DNA polyermase are used to isolate a full-length DNA construct. However, oligonucleotides can incorrectly hybridize resulting in incorrect extensions. In this situation, it is likely that the combined oligonucleotides will not produce the desired gene.

Therefore, a need exists to provide methods and compositions to improve the synthesis of mutated genes.

### SUMMARY OF THE INVENTION

Provided herein are methods of recombining nucleotide sequences and related compositions. Historically, nucleotide sequences, particularly polypeptide-encoding nucleotide sequences, have been mutated or combined with other nucleotide sequences to provide new properties or encode polypeptides possessing new properties. Heretofore, methods of mutating or combining nucleotide sequences have been limited by the molecular techniques available for manipulation of nucleic acid molecules. Provided herein are methods for manipulating nucleotide sequences that permit greater control of sequence recombination compared to traditional methods.

Provided herein are compositions, comprising a set of oligonucleotides configured to assemble into a group of non-overlapping polypeptide-encoding synthetic polynucleotides, wherein the oligonucleotides of the set have been mutually and globally thermodynamically optimized by computerized analysis, such that when Tmc represents the melting temperature of a correct hybridization between a given possible nucleotide internal sequence IS of length n and a fully complementary nucleotide sequence thereto ISC of length n, wherein n is selected to be at least 10; and when Tmi represents the highest melting temperature of any possible incorrect hybridization between that same ISC and any other oligonucleotide of the set, or portion thereof; there exists a temperature gap such that for each possible IS and corresponding ISC of the set, Tmc is higher than Tmi. In some such compositions, the group comprises at least 2 polynucleotides. In some such compositions, the group comprises at least 3 polynucleotides. In some such compositions, the group comprises at least 4 polynucleotides. In some such compositions, the group comprises at least 5 polynucleotides. In some such compositions, for all oligonucleotides of a selected subset of the entire set, the lowest Tmc of any fully complementary IS/ISC pair is higher than the highest Tmi associated with any ISC within the subset. In some such compositions, for all oligonucleotides of a first selected subset of the entire set, the lowest Tmc of any fully complementary IS/ISC pair is higher than the highest Tmi associated with any ISC within the first subset, and for all oligonucleotides of a second selected subset of the entire set, the lowest Tmc of any fully complementary IS/ISC pair is higher than the highest Tmi associated with any ISC within the second subset. In some such compositions, the oligonucleotides of said first subset are non-overlapping. In some such compositions, the oligonucleotides of said second subset are non-overlapping. In some such compositions, for all oligonucleotides of the entire set, the lowest Tmc of any fully complementary IS/ISC pair is higher than the highest Tmi associated with any ISC. In some such compositions, n is at least 15. In some such compositions, n is at least 20. In some such compositions, each polynucleotide in the group encodes at least a portion of a protein from the same protein family or superfamily.

Also provided are compositions comprising a set of oligonucleotides configured to assemble into a group of polynucleotides, each encoding a desired polypeptide; and a primer having a first region that is fully complementary to a sequence S1 of a first polynucleotide of the polynucleotide group, wherein sequence S1 is of a minimum length of about 5 bases and having a second region that is fully complementary to a sequence S2 of a second polynucleotide of the polynucleotide group, wherein sequence S2 is of a minimum length of about 5 bases; wherein codons of the oligonucleotides of the oligonucleotide set have been selected from among synonymous codons, and as a result, the melting temperature of the hybridization of the first region of the first primer to S1 is greater than the melting temperature of any incorrect hybridization of the first region to any other sequence of the set and the melting temperature of the hybridization of the second region of the second primer to S2 is greater than the melting temperature of any incorrect hybridization of the second region to any other sequence in the set. In some such compositions, S1 and S2 are of minimum length of about 10 bases. In some such compositions, the lengths of S1 and S2 are between about 9 and about 13 bases.

Also provided are compositions, comprising a set of oligonucleotides configured to assemble into a group of polynucleotides, each encoding a desired polypeptide; and a primer pair, comprising a first and a second primer, wherein the first primer has a first region that is fully complementary to a sequence S1 of a first polynucleotide of the polynucleotide group, wherein sequence S1 is of a minimum length of about 5; the second primer has a second region that is fully complementary to a sequence S2 of a second polynucleotide of the polynucleotide group, wherein sequence S2 is of a minimum length of about 5; and the first primer has a third region that is identical to, or fully complementary to, a fourth region of the second primer, wherein the third region of the first primer includes none, part, or all of the first region of the first primer, and the fourth region of the second primer includes none, part, or all of the second region of the second primer; and wherein codons of the oligonucleotides of the oligonucleotide set have been selected from among synonymous codons, and as a result, the melting temperature of the hybridization of the first region of the first primer to S1 is greater than the melting temperature of any incorrect hybridization of the first region to any other sequence of the set and the melting temperature of the hybridization of the second region of the second primer to S2 is greater than the melting temperature of any incorrect hybridization of the second region to any other sequence in the set. In some such compositions, S1 and S2 are of minimum length of about 10 bases. In some such compositions, the lengths of S1 and S2 are between about 9 and about 13 bases. In some such compositions, the concentration of the primer pair is greater than the concentration of any oligonucleotide of any given sequence. In some such compositions, the concentration of the primer pair is at least five times greater than the concentration of any oligonucleotide of any given sequence. In some such compositions, the third region of the first primer comprises a portion less than the entirety of the first region of the first primer and/or the fourth region of the second primer comprises a portion less than the entirety of the second region of the second primer. In some such compositions, the third region of the first primer comprises all of the first region of the first primer and the fourth region of the second primer comprises all of the second region of the second primer. In some such compositions, the third region of the first primer comprises one or more bases not identical to or not complementary to S2 and the fourth region of the second primer comprises one or more bases not identical to or not complementary to S1.

Also provided are methods for creating a chimeric polynucleotide from a set of oligonucleotides configured to assemble into a group of polynucleotides, comprising providing a set of oligonucleotides as provided herein; providing at least one primer, said primer having a first region uniquely complementary to a sequence of a first polynucleotide in the group and a second region uniquely complementary to a second polynucleotide of the group, and combining the primer with an oligonucleotide or polynucleotide comprising the first region of said first polynucleotide and an oligonucleotide or polynucleotide comprising the second region of said second polynucleotide; and PCR amplifying to create a chimeric polynucleotide having some sequence from the first polynucleotide and some sequence from the second polynucleotide. In some such methods, said primer is between about 18 and about 25 bases in length. In some such methods, the concentration of the primer is greater than the concentration of any oligonucleotide of any given sequence. In some such methods, the concentration of the primer is at least five times greater than the concentration of any oligonucleotide of any given sequence.

Also provided are methods for creating a plurality of chimeric polynucleotides, respectively encoding a plurality chimeric polypeptides, comprising providing a set of oligonucleotides as provided herein; providing a plurality of different primers, each said primer having a first region uniquely complementary to a sequence of one of the polynucleotides of the group and a second region uniquely complementary to a sequence of another of the polynucleotides of the group, and, optionally, a third region not complementary to any polynucleotides of the group, wherein the plurality of primers differ from each other in at least the first region, the second region, or the third region; contacting each of the primers with an oligonucleotide or polynucleotide complementary to the first or second regions to form primer-oligonucleotide or primer-polynucleotide hybridizations; and PCR extending the hybridized primers to create chimeric polynucleotides. In some such methods, the group of polynucleotides contains at least 3 different polynucleotides and wherein each of the primers is contacted with assembled polynucleotides of the group. In some such methods, each of the primers is simultaneously contacted with assembled polynucleotides of the group. In some such methods, the group of polynucleotides is serially contacted with different pluralities of primers. In some such methods, each of the primers is simultaneously contacted with assembled oligonucleotides of the set. In some such methods, the set of oligonucleotides is serially contacted with different pluralities of primers.

Also provided herein are primers comprising a first region that is fully complementary to a sequence S1 of a first oligonucleotide of the oligonucleotide set provided herein, wherein sequence S1 is of a minimum length of about 5 bases, said primer further comprising a second region that is fully complementary to a sequence S2 of a second oligonucleotide of the oligonucleotide set, wherein sequence S2 is of a minimum length of about 5 bases, wherein the melting temperature of the hybridization of the first region to S1 is greater than the melting temperature of any incorrect hybridization of the first region to any other sequence of the set and the melting temperature of the hybridization of the second region to S2 is greater than the melting temperature of any incorrect hybridization of the second region to any other sequence in the set.

Also provided are primer pairs, comprising a first and a second primer, wherein the first primer has a first region that is fully complementary to a sequence S1 of a first oligonucleotide of the oligonucleotide set provided herein, wherein sequence S1 is of a minimum length of about 5; the second primer has a second region that is fully complementary to a sequence S2 of a second oligonucleotide of the oligonucleotide set, wherein sequence S2 is of a minimum length of about 5; and the first primer has a third region that is identical to, or fully complementary to, a fourth region of the second primer, wherein the melting temperature of the hybridization of the first region of the first primer to S1 is greater than the melting temperature of any incorrect hybridization of the first region to any other sequence of the set and the melting temperature of the hybridization of the second region of the second primer to S2 is greater than the melting temperature of any incorrect hybridization of the second region to any other sequence in the set.

### BRIEF DESCRIPTION OF THE DRAWINGS

FIG. 1A illustrates an embodiment for the synthesis a DNA sequence by overlap extension in which a medium-sized piece of DNA is divided into 12 short segments.

FIG. 1B illustrates an embodiment of the disclosed method for synthesizing a DNA sequence by overlap extension in which a large piece of DNA is divided into five medium-sized pieces.

FIG. 2 illustrates an embodiment using a direct self-assembled DNA construct, from which a full-length DNA sequence is produced.

FIG. 3 illustrates an embodiment of the disclosed method for synthesizing a synthetic gene or piece of DNA.

FIG. 4A-4C illustrate point mutations, regional mutations, and directed shuffling, respectively.

FIGS. 5A-5C illustrate the yields of full-length oligonucleotide of length 20 to 250 nt, for coupling efficiencies of 99.5%, 99%, and 98%, respectively.

FIG. 6 illustrates formation of oligonucleotides and their assembly to the desired gene. (A) schematically illustrates an embodiment comprising division and reassembly of a gene from intermediate fragments. (B) schematically illustrates an embodiment comprising the division and reassembly of one of the intermediate fragments into oligonucleotides that include leader and trailer sequences. (C) schematically illustrates an embodiment of a leader used in the expression of a polypeptide from an intermediate fragment. (D) schematically illustrates an embodiment of a trailer used in the expression of a polypeptide from an intermediate fragment.

FIGS. 7A and 7B are probability distributions of theoretical melting temperatures of oligonucleotides and intermediate fragments.

FIG. 8 diagrams a hierarchical gene assembly process.

FIG. 9 is an electrophoretogram of assembly of intermediate DNA fragments of the Ty3 IN gene comprised either of optimized oligonucleotides (FIG. 9A) or of un-optimized oligonucleotides (FIG. 9B).

FIG. 10 is an electrophoretogram of assembly of DNA gene fragments.

FIG. 11 shows DNA fragment rearrangement.

FIG. 12A illustrates gene synthesis using point mutations and DNA fragments and FIG. 12B is an electrophoretogram of assembly of intermediate DNA fragments of the p53 gene.

FIG. 13 illustrates directed shuffling of DNA sequences among the Integrase genes of Ty3, MLV and HIV-1.

### DETAILED DESCRIPTION OF THE PREFERRED EMBODIMENT

Provided herein are methods of recombining nucleotide sequences and related compositions. Historically, nucleotide sequences, particularly polypeptide-encoding nucleotide sequences, have been mutated or combined with other nucleotide sequences to provide new properties or encode polypeptides possessing new properties. Heretofore, methods of mutating or combining nucleotide sequences have been limited by the molecular techniques available for manipulation of nucleic acid molecules. Provided herein are methods for manipulating nucleotide sequences that permit greater control of sequence recombination compared to traditional methods.

For example, provided are methods for contacting one or more primers with a set of oligonucleotides configured to assemble into a group of non-overlapping polynucleotides, to form one or more chimeric polynucleotides having portions of nucleotide sequence from two or more polynucleotides of the group of non-overlapping polynucleotides. The primers and set of oligonucleotides are configured in such a manner as to decrease the likelihood of unintended hybridization events relative to intended hybridization events such that manipulation of hybridization conditions favor only intended hybridization events. For example, in one such embodiment, a plurality of non-overlapping polypeptide-encoding synthetic polynucleotides possess nucleotide sequences that have been mutually and globally optimized for correct overlap hybridization between a given nucleotide internal sequence (IS) and a fully complementary nucleotide sequence (ISC) and methods of making and using thereof. In this manner, various aspects of nucleotide sequence recombination can be precisely controlled. The methods can be used to provide a large, diverse, and/or specific library of desired polynucleotides including, if desired, various specific sequence substitutions, insertions or deletions. The library can be further used in screens or selections to determine specific sequences that produce proteins with a desired property (e.g., enzymatic activity, binding properties, and solubility). Additional related methods and compositions also are provided herein, as described in more detail below.

### Methods

In some embodiments, the present invention relates to a method comprising (a) optimizing sequences in component oligonucleotides of a group of polynucleotides to facilitate preferential hybridization, and (b) achieving a DNA melting temperature gap between correct (high melting temperature) and incorrect (low melting temperature) hybridizations of the oligonucletides. Some methods relate to methods for creating one or more chimeric polynucleotides from a group of polynucleotides or mutant genes, or from a set of oligonucleotides configured to assemble into a group of polynucleotides. In some embodiments, oligonucleotides are starting materials for assembling into a group of polynucleotides. Typically, the oligonucleotides are single-stranded DNA pieces. The oligonucleotides may be, for example, at least about 10, at least about 20, at least about 30, at least about 40, at least about 50, at least about 75, at least about 100, at least about 120, at least about 140, at least about 160, at least about 180 or at least about 200 bases long. The oligonucleotides may be, for example, less than about 1000, less than about 500, less than about 300, less than about 200, less than about 180, less than about 160, less than about 140, less than about 120, less than about 100, less than about 80, less than about 60, less than about 50, less than about 40 or less than about 20 bases long. The oligonucleotides may be mutually and globally thermodynamically optimized by, for example, computer analysis methods known in the art, such as those described in U.S. Patent Application 2005/0106590, which is incorporated by reference herein in its entirety. A set of oligonucleotides can be non-overlapping or substantially non-overlapping, such that the oligonucleotides within the set are not configured to directly hybridize with each other (e.g., without the use of a primer or another oligonucleotide).

A polynucleotide can comprise one or more oligonucleotides. A group of polynucleotides can comprise one or more sets of oligonucleotides. A polynucleotide can comprise one or more sequences encoding at least one desired polypeptide. A desired polypeptide can include, for example, enzymes, antibodies, hormones and hormone receptors. For example, a desired polypeptide can be a mammalian enzyme such as bovine chymosin, human tissue plasminogen activator etc., a mammalian hormone such as human growth hormone, human interferon, human interleukin, or other mammalian protein such as human serum albumin. A desired polypeptide can also be a bacterial enzyme such as α-amylase from Bacillus species, lipase from Pseudomonas species, etc. A desired polypeptide can be a fungal enzyme such as lignin peroxidase or Mn²⁺-dependent peroxidase from Phanerochaete, glucoamylase from Humicola species and aspartyl protease from Mucor species. Any of a variety of additional desired polypeptides will be readily apparent to those skilled in the art. Non-encoding sequences may also be included in the polynucleotides and/or oligonucleotides. Such non-encoding sequences can include, for example, an intron or other splicing-related sequence, a transcriptional regulatory sequence or a translational regulatory sequence.

A polynucleotide also can encode a non-translated nucleic acid molecule, such as, for example, tRNA, rRNA, siRNA, or any of a variety of structural DNA sequences, such as histone-binding DNA.

The methods provided herein can be used to generate a set of oligonucleotides configured to assemble into a group of polypeptide-encoding synthetic polynucleotides, such as, for example, non-overlapping polypeptide-encoding synthetic polynucleotides, wherein the oligonucleotides of the set have been mutually and globally thermodynamically optimized such that: when Tmc represents the melting temperature of a correct hybridization between a given possible nucleotide internal sequence IS of length n and a fully complementary nucleotide sequence thereto ISC of length n; and when Tmi represents the highest melting temperature of any possible incorrect hybridization between that same ISC and any other oligonucleotide of the set, or portion thereof; there exists a temperature gap such that for each possible IS and corresponding ISC of the set, Tmc is higher than Tmi. In such methods, n can be at least 3, 4, 5, 6, 7, 8, 9, 10, 11, 12, 13, 14, 15, 16, 17, 18, 19, 20, 22, 24, 26, 28, 30, 32, 36, 38, or 40 bases.

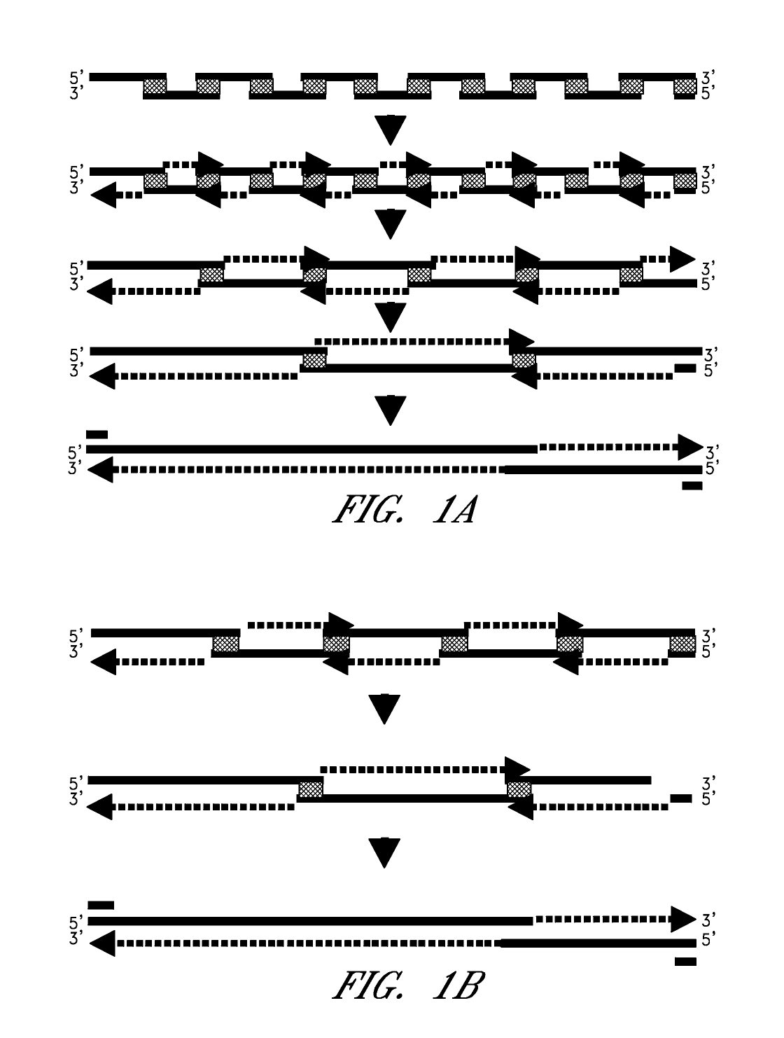

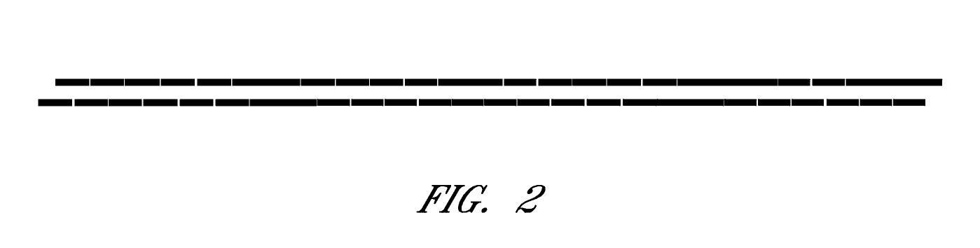

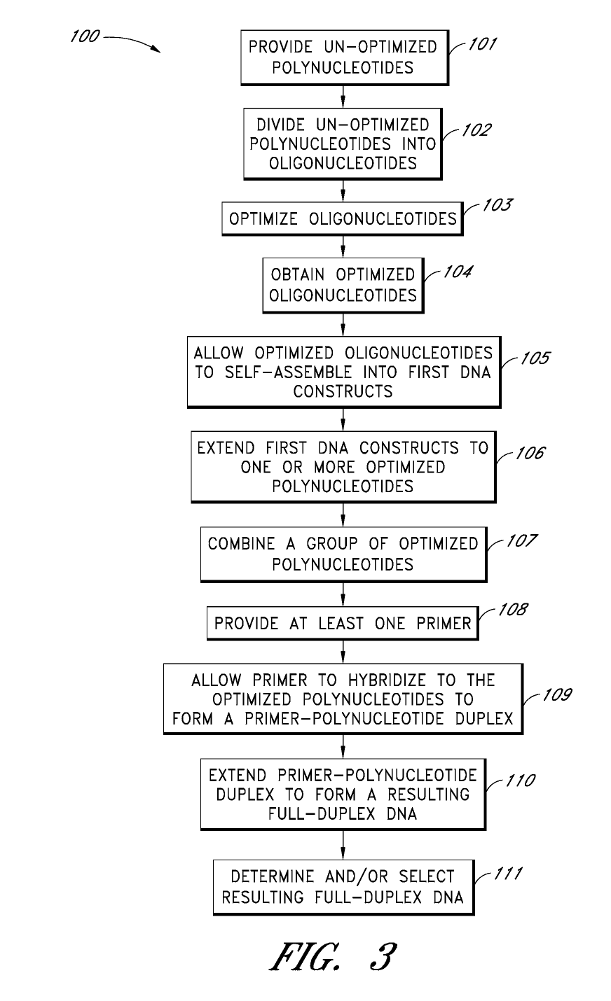

In some embodiments, methods disclosed herein to optimize sequences in the component oligonucleotides of a plurality of first polynucleotides to facilitate preferential hybridization can be combined with a divide-and-conquer DNA synthesis method (see Aho et al. The Design and Analysis of Computer Algorithms Addison-Wesley; Reading, Mass.: 1974 and U.S. Patent Application 2005/0106590, both of which are incorporated by reference in their entireties). A long DNA sequence or full-length gene can be broken recursively into smaller overlapping pieces. The smaller overlapping pieces may be synthesized, and the DNA sequence or gene can then be reassembled in the reverse order of the disassembly. A reassembly step may be performed by overlap extension using a high-fidelity DNA polymerase as illustrated in FIG. 1A and FIG. 1B, or by ligation as illustrated in FIG. 2. Those skilled in the art will realize that ligation can also be used to reassemble a single strand of DNA using a variation of the DNA construct illustrated in FIG. 2 in which the pieces of DNA that comprise one strand abut, but all of the pieces of DNA comprising the complementary strand do not. Another method for reassembly is cloning into an expression vector and transformation of an appropriate host. Those skilled in the art will understand that many methods of cloning are compatible with the disclosed method, for example, exonuclease III cloning, topoisomerase cloning, restriction enzyme cloning, and homologous recombination cloning.

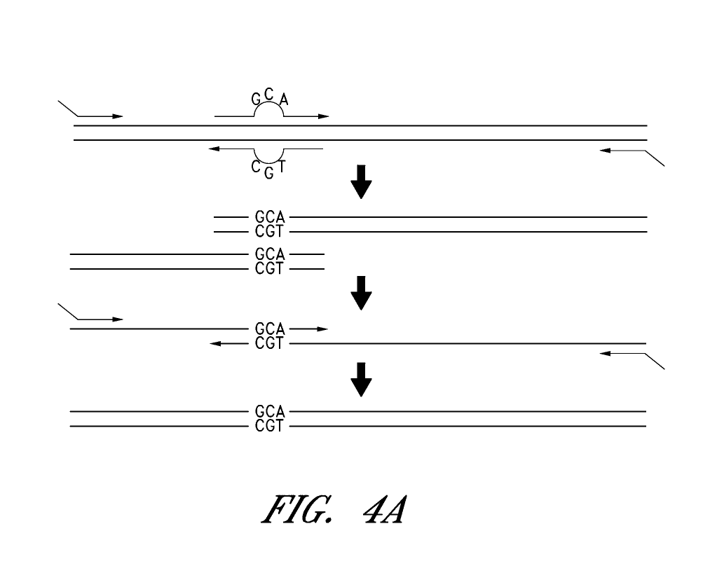

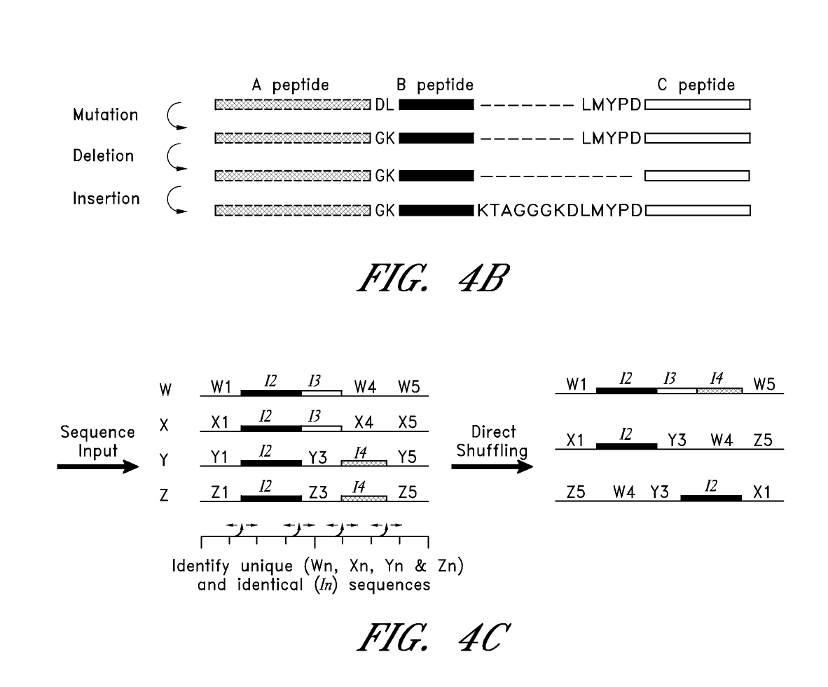

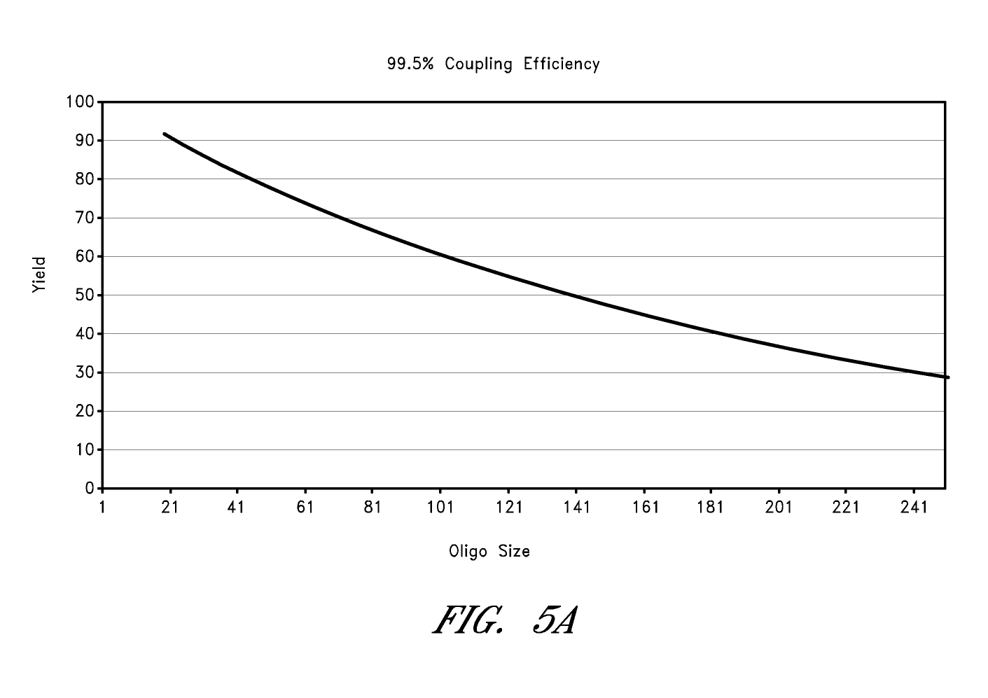

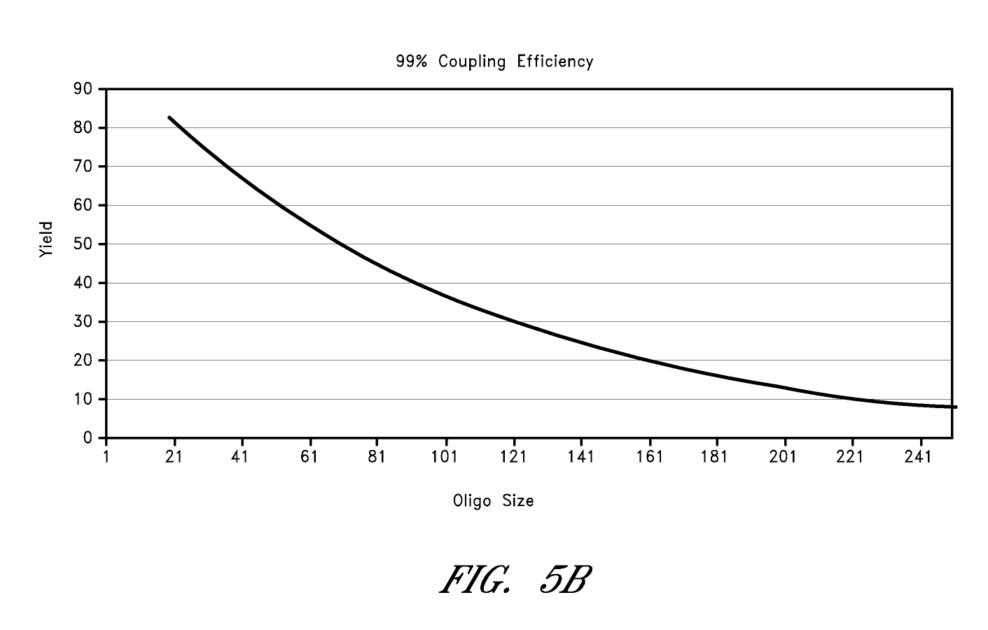

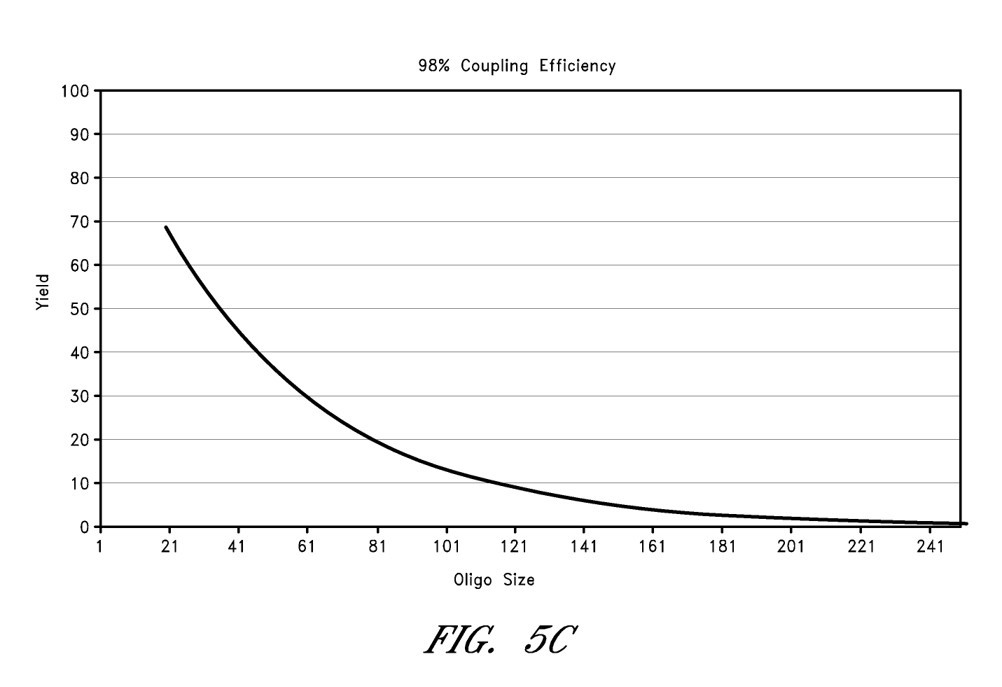

One embodiment of the methods provided herein can comprise a design process and an assembly process. In one embodiment, the synthetic gene is designed and assembled according to a method illustrated as method 100 in FIG. 3. In step 101, a plurality of initial un-optimized polynucleotides is provided. In step 102, the plurality of initial un-optimized polynucleotides is divided into small pieces of DNA or oligonucleotides. In step 103, the oligonucleotides are optimized. In step 104, the optimized oligonucleotides of DNA are obtained. In step 105, oligonucleotides of each polynucleotide are allowed to self-assemble into first DNA constructs. In step 106, the first DNA constructs are extended to a first set of optimized polynucleotides.

The assembled optimized polynucleotides can be used in subsequent methods, as exemplified in steps 107-111 of FIG. 3. In step 107, a plurality of optimized polynucleotides is combined. In step 108, a pair of primers is provided. In step 109, primers hybridize to optimized polynucleotides to form primer-polynucleotide duplexes. In step 110, the primer-polynucleotide duplex is extended to a resulting full-duplex DNA. In step 111, a property indicative of the likelihood of the correctness of the resulting full-duplex DNA is determined, and pieces of DNA that are likely to have the correct sequence selected.

Another embodiment of the methods provided herein can comprise a design process. In one embodiment, the synthetic gene is designed and assembled according to a modification of the method illustrated in FIG. 3. In step 101, a plurality of initial un-optimized polynucleotides is provided. In step 102, the plurality of initial un-optimized polynucleotides is divided into small pieces of DNA or oligonucleotides. In step 103, the oligonucleotides are optimized. In step 104, the optimized oligonucleotides of DNA are obtained.

The obtained oligonucleotides can be used in subsequent methods, such as a modification of the method exemplified in steps 107-111 of FIG. 3. In a first step, a plurality of optimized oligonucleotides is combined. In a second step, a primer is provided. In a third step, primers hybridize to an optimized oligonucleotide to form a primer-oligonucleotide duplex. In a fourth step, the primer-oligonucleotide duplex is extended to a resulting full-duplex DNA. In a fifth step, a property indicative of the likelihood of the correctness of the resulting full-duplex DNA is determined, and pieces of DNA that are likely to have the correct sequence selected.

### Step 101: A Plurality of Initial Un-Optimized Polynucleotides is Provided

In some embodiments, one or more polynucleotides of the plurality of initial un-optimized polynucleotides are a fragment of DNA. In some embodiments, each polynucleotide of the plurality of initial un-optimized polynucleotides is a DNA fragment. The DNA fragment may be a fragment of a DNA sequence, which may be, for example, the DNA sequence of a gene or a cDNA sequence. A DNA fragment may be, by way of non-limiting example, about 1,500 bases long. The polynucleotides may be different fragments of one DNA sequence of a gene. The plurality of initial un-optimized polynucleotides may or may not contain all DNA fragments of the DNA sequence of a gene. In some embodiments, the plurality of initial un-optimized polynucleotides comprises non-adjacent fragments of the DNA sequence of a gene. The polynucleotides may be various mutations of the same DNA fragment. In some embodiments, the polynucleotides are DNA sequences of a gene.

In some embodiments, one or more polynucleotides of the plurality of initial un-optimized polynucleotides is a DNA fragment, wherein the DNA fragment can be combined with other DNA fragments to form larger DNA sequences, which may be the DNA sequence of a gene. For example, a DNA fragment may reassemble with other DNA fragments by ligation. In another example, a DNA fragment may reassemble with other DNA fragments by overlap extension. In yet another example, a large piece of DNA is reassembled by cloning in an expression vector.

In some embodiments, the group of polynucleotides can include polynucleotides that are evolutionarily related. For example, polynucleotides can be selected from different organisms, such as organisms from different domains, kingdoms, phyla, divisions, classes, orders, families, genera, species, subspecies, and/or strains, where the polynucleotides from the various selected organisms are identified as phylogenetically related. In embodiments where polynucleotides are selected from different organisms, the group of polynucleotides can contain polynucleotides from at least 2, 3, 4, 5, 6, 7, 8, 9, 10, 12, 15, 20, 25, 30, 35, 40, 45, 50 or more different organisms selected from different domains, kingdoms, phyla, divisions, classes, orders, families, genera, species, subspecies, and/or strains. In embodiments where polynucleotides are selected from different organisms, the group of polynucleotides can contain polynucleotides from at least 3, 4, 5, 6, 7, 8, 9, 10, 12, 15, 20, 25, 30, 35, 40, 45, 50 or more different organisms whose closest relatedness falls into least two taxonomic categories selected from different domains, kingdoms, phyla, divisions, classes, orders, families, genera, species, subspecies, and/or strains. Phylogenetic relatedness of biomolecules between organisms can be determined according to any of a variety of methods known in the art, including, but not limited to, computational/statistical methods, known phylogenetic relation databases, structural classification methods, and the like. In some embodiments, the group of polynucleotides can include polynucleotides or polynucleotide-encoded polypeptides from the same structural class, superfamily, family or subfamily. The meaning of polynucleotide or polypeptide structural class, superfamily, family or subfamily is in accordance with that commonly used in the art, for example, Structural Classification of Proteins (SCOP). See Murzin A. G., Brenner S. E., Hubbard T., Chothia C. (1995) SCOP: a structural classification of proteins database for the investigation of sequences and structures. J. Mol. Biol. 247, 536-540; Lo Conte L., Brenner S. E., Hubbard T. J. P., Chothia C., Murzin A. (2002) SCOP database in 2002: refinements accommodate structural genomics. Nucl. Acid Res. 30(1), 264-267; Andreeva A., Howarth D., Brenner S. E., Hubbard T. J. P., Chothia C., Murzin A. G. (2004) SCOP database in 2004: refinements integrate structure and sequence family data. Nucl. Acid Res. 32:D226-D229; Andreeva A., Howorth D., Chandonia J.-M., Brenner S. E., Hubbard T. J. P., Chothia C., Murzin A. G. (2008) Data growth and its impact on the SCOP database: new developments. Nucl. Acid Res. 36: D419-D425, all of which are herein incorporated by reference in their entireties. Any amount of diversity or relatedness of the evolutionarily related polynucleotides can be selected, according to the level of diversity desired in the group of polynucleotides. In another example, evolutionarily related polynucleotides from a single organism also can be included in the group of polynucleotides.

The group of polynucleotides can include two or more polynucleotides that possess a designated level of sequence conservation. For example, the group of polynucleotides can comprise two or more polynucleotides that each possess at least, or at least about, 5%, 6%, 7%, 8%, 9% 10%, 12%, 15%, 20%, 25%, 30%, 35%, 40%, 45%, 50%, 60%, 70%, 80%, or 90% sequence identity to at least one other polynucleotide of the group of polynucleotides. In such embodiments, the number of sequence-conserved polynucleotides in the group of polynucleotides can be at least 2, 3, 4, 5, 6, 7, 8, 9, 10, 12, 15, 20, 25, 30, 35, 40, 50, 75, 100, 150 or more polynucleotides.

In some embodiments, the group of polynucleotides comprise or consist of non-related polypeptide-encoding polynucleotides. The methods provided herein permit recombination of nucleotide sequences between sequences completely lacking any sequence homology. Thus, in contrast to traditional shuffling methodologies, there is no sequence homology dependence of the recombination as is present in, for example, homologous recombination-based shuffling methodologies. For example, the group of polypeptides can comprise or consist of polynucleotides that are not evolutionarily related to at least one other polynucleotide of the group of polynucleotides. In another example, the group of polynucleotides can comprise or consist of polynucleotides that are not evolutionarily related to any other polynucleotide of the group of polynucleotides. In embodiments where polynucleotides are not evolutionarily related, the group of polynucleotides can contain polynucleotides from no more than 2, 3, 4, 5, 6, 7, 8, 9, 10, 12, 15, 20, 25, 30, 35, 40, 45, 50 or more different organisms selected from different domains, kingdoms, phyla, divisions, classes, orders, families, genera, species, subspecies, and/or strains.

The group of polynucleotides can include two or more polynucleotides that possess a designated level of sequence differences. For example, the group of polynucleotides can comprise or consist of two or more polynucleotides that each possess no more, or no more than about, 1%, 2%, 3%, 4%, 5%, 6%, 7%, 8%, 9% 10%, 12%, 15%, 20%, 25%, 30%, 35%, 40%, 45% or 50% sequence identity to at least one other polynucleotide of the group of polynucleotides. In another example, the group of polynucleotides can comprise or consist of two or more polynucleotides that each possess no more, or no more than about, 1%, 2%, 3%, 4%, 5%, 6%, 7%, 8%, 9% 10%, 12%, 15%, 20%, 25%, 30%, 35%, 40%, 45% or 50% sequence identity to any other polynucleotide of the group of polynucleotides. In such embodiments, the number of sequence-conserved polynucleotides in the group of polynucleotides can be at least 2, 3, 4, 5, 6, 7, 8, 9, 10, 12, 15, 20, 25, 30, 35, 40, 50, 75, 100, 150 or more polynucleotides.

One or more of the plurality of initial un-optimized polynucleotides may be synthetic polynucleotides. The plurality of initial un-optimized polynucleotides may be polypeptide-encoding polynucleotides. The plurality of initial un-optimized polynucleotides may be non-overlapping polynucleotides. In some embodiments, the plurality of initial un-optimized polynucleotides is a plurality of non-overlapping polypeptide-encoding synthetic polynucleotides.

In some embodiments, non-overlapping polynucleotides can be defined as polynucleotides not containing overlap regions complementary to overlap regions of other polynucleotides of the plurality of initial un-optimized polynucleotides. In some embodiments, non-overlapping polynucleotides can be defined as polynucleotides not containing overlap regions complementary to regions of polynucleotides of which they will be combined with in step 106. In some embodiments, non-overlapping polynucleotides can refer to polynucleotides not having overlap regions.

In some embodiments, the plurality of initial un-optimized polynucleotides can be overlapping polynucleotides. The plurality of initial un-optimized polynucleotides may comprise overlap regions that are, for example, at least the width of a restriction site (typically from about four to about six bases) or, for example, from about 25 to about 30 bases or greater. In some embodiments, the plurality of initial un-optimized polynucleotides comprises adjacent fragments of the DNA sequence of a gene.

In some embodiments, the plurality of initial un-optimized polynucleotides comprises at least at least 2, 3, 4, 5, 6, 7, 8, 9, 10, 12, 15, 20, 25, 30, 35, 40, 50, 75, 100, 150 or more polynucleotides.

In some embodiments, the plurality of initial un-optimized polynucleotides is a single long DNA sequence and/or DNA sequence of a gene, which, after optimization, will be divided into a plurality of polynucleotides.

Step 102: The Initial Un-Optimized Polynucleotides are Divided into Oligonucleotides.

The initial un-optimized polynucleotides are divided into overlapping short pieces of DNA that are readily obtainable—that is, small pieces or segments. Preferably, each short piece is small enough to be synthesized readily. In a preferred embodiment, the un-optimized polynucleotides are designed for “direct self-assembly.” In this embodiment, each un-optimized polynucleotides divided into, by way of non-limiting example, from about 50 to about 60 overlapping small pieces of from about 50 to about 60 bases or fewer. Preferably, the adjacent small pieces of DNA from the same strand abut, i.e., hybridize to form first DNA constructs with no gaps between the pieces. The polynucleotides can be reassembled by ligation (“direct self-assembly and ligation”). In an embodiment in which adjacent small pieces from the same strand do not abut, i.e., hybridize to form first DNA constructs with single-stranded gaps between the double-stranded overlaps, the polynucleotides can be reassembled by overlap extension. In another embodiment, adjacent small pieces from the same strand abut, i.e., hybridize to form first DNA constructs with no single-stranded gaps between the double-stranded overlaps, and the polynucleotides reassembled by overlap extension. In another embodiment, first DNA constructs with a combination of gaps and no gaps is reassembled by overlap extension. In another embodiment, the polynucleotides are reassembled by cloning in an expression vector. In this embodiment, the ends of the first DNA constructs may have any combination of gaps and no gaps. Preferably, the ends of the large piece of DNA are adapted for insertion into an expression vector, for example, complementary to a restriction site in the expression vector.

In some embodiments, the initial un-optimized polynucleotides are DNA fragments from DNA sequences of one or more genes. “Hierarchical assembly” or “recursive assembly” refers to assembling polynucleotides corresponding to DNA fragments from oligonucleotides and forming long DNA sequences, such as that of a gene, from the DNA fragments. In these embodiments, a large piece of DNA may divided first into about three to about ten medium-sized pieces of DNA or DNA fragments, preferably, about five to about seven pieces. Each DNA fragment is then subdivided into overlapping oligonucleotides, preferably, from about six to about 12 pieces. As described above for direct self-assembly, the DNA pieces at each level of recursion may be designed for reassembly by any combination of methods, including ligation, overlap extension, or cloning. In a preferred embodiment, the DNA pieces are reassembled by overlap extension.

### Step 103. The Oligonucleotides are Optimized.

The sequences in the component oligonucleotides of a plurality of polynucleotides may be mutually and/or globally optimized. The sequences may be optimized simultaneously. The sequences may be optimized by a computerized analysis.

The plurality of polynucleotides may be all of the initial un-optimized polynucleotides. The plurality of polynucleotides may be all of the polynucleotides combined together in step 106.

In some embodiments, the optimization may comprise thermodynamic optimization. The thermodynamic optimization may comprise optimizing sequences of polynucleotides, where the result of the optimization is that internal sequences of the polynucleotides are uniquely thermodynamically addressable. The thermodynamic optimization may comprise optimizing internal sequences such that a primer can be created for each internal sequence that is preferentially complementary to that internal sequence over all other internal sequences in the plurality of polynucleotides.

In some embodiments, oligonucleotides of the plurality of polynucleotides are mutually and globally optimized by computer analysis and, optionally each codon is selected, such that when Tmc represents a melting temperature of a correct hybridization between a given possible nucleotide internal sequence IS of length n and a fully complementary nucleotide sequence thereto ISC of length n, and when Tmc represents the highest melting temperature of any possible incorrect hybridization between that same ISC and any other portion of the polynucleotides of the plurality of polynucleotides, there exists a temperature gap such that for each possible IS and corresponding ISC of the set, Tmc is higher than Tmi. In some embodiments, n is selected to be at least 3, 4, 5, 6, 7, 8, 9, 10, 11, 12, 13, 14, 15, 16, 17, 18, 19, 20, 22, 24, 26, 28, 30, 32, 36, 38, 40, 50, 60, 70, 80, 90 or 100 bases. In some embodiments, n is selected to be greater than 8 bases. A nucleotide internal sequence may include a sequence of an oligonucleotide or polynucleotide that does not include a start or stop codon. In some embodiments, for all oligonucleotides of the set of oligonucleotides, the lowest Tmc of any fully complementary IS/ISC pair is higher than the highest Tmi associated with any ISC.

In some embodiments, the optimization can improve, or maximize, the probability that a given first nucleotide internal sequence hybridizes to a second nucleotide sequence fully complementary thereto. In some embodiments, the optimization can improve, or maximize, the probability that a plurality of given first nucleotide internal sequences hybridizes to a specific plurality of second complementary nucleotide internal sequences. The hybridization of the plurality of first internal sequences to the plurality of second complementary internal sequences may provide a single DNA sequence, which may be, for example, the DNA sequence of a gene. In some embodiments, the optimization can improve, or maximize, the probability that the oligonucleotides hybridize to each other such that assembly methods can be used to form the selected group of polynucleotides without significantly forming polynucleotides with unintended sequences.

Some embodiments use no or limited sequence optimization. For example, changes in a nucleotide sequence can change the secondary structure of DNA and RNA. Changes in the secondary structure in RNA viral genomes can affect the viability of the viruses. In some embodiments, no sequence optimization is performed. In other embodiments, selected sequences are optimized as described herein and other sequences are not.

In some embodiments, the division described in step 102 is optimized to increase the probability that the pieces of DNA will reassemble into the desired DNA sequence. The boundary points between adjacent pieces of DNA are adjusted to create or to increase a temperature gap, or to disrupt other incorrect hybridizations, for example, hairpins.

Optimization can comprise identifying optimized nucleic acid sequences of oligonucleotides that can be assembled to polynucleotide encoding a polypeptide. Each amino acid of a protein is coded by a codon comprising three nucleotides. The genetic code is degenerate, meaning that there is a plurality of codons that can code for the same protein. Still, polynucleotides consisting of different codons encoding the same protein can differ in other properties, such as their melting temperatures. By optimizing the nucleic acid sequences of the oligonucleotides of a plurality of polynucleotides, the melting temperature of a correct hybridization between a given internal nucleotide sequence and complementary nucleotide sequence can be higher, and in some embodiments substantially higher, than the melting temperature of an incorrect hybridization between that same given internal nucleotide sequence and any other portion of the group of polynucleotides. Other sequence properties in addition to codon identity can affect the hybridization of an internal nucleotide sequence with another sequence. For example, the codon context, or the nucleic acids comprising the surrounding codons can affect the hybridization, for example via enhanced base stacking interactions. In some embodiments, optimization of nucleic acid sequences includes optimizing codon usage and other sequence properties, such as, for example, codon context, such as, for example, codon pair usage. A DNA sequence in a regulatory region may be optimized by taking advantage of the degeneracy in the regulatory region consensus sequence. A DNA sequence outside a coding or regulatory region, i.e., in an intergenic region, may be optimized by direct base assignment.

A broad temperature gap between the highest-melting incorrect hybridization and the lowest-melting correct hybridization means that, with high probability, at higher temperatures, most incorrect hybridizations have melted and most correct hybridizations have annealed. In optimizing the base sequences, theoretical melting temperatures can be calculated for all possible correct and incorrect hybridizations by methods known in the art, for example, using Mfold. Such methods are disclosed, for example, in M. Zuker et al. “Algorithms and thermodynamics for RNA secondary structure prediction: A practical guide.” in RNA Biochemistry and Biotechnology, Barciszewsld & Clark, eds.; Kluwer: 1999; D. H. Mathews et al. “Expanded sequence dependence of thermodynamic parameters provides robust prediction of RNA secondary structure” J. Mol. Biol., 1999, 288, 910-940; J. Santa-Lucia “A unified view of polymer, dumbbell, and oligonucleotide DNA nearest-neighbor thermodynamics” Proc. Natl. Acad. Sci. USA, 1998, 95, 1460-1465; the disclosures all of which are incorporated by reference in their entireties. The figure of merit is the gap between the lowest-melting correct match and the highest-melting incorrect match. Examples of incorrect matches can include: (a) hairpins, in which a short segment folds back and hybridizes to itself; (b) dimers, in which a short segment is partially self-complementary; (c) intersegment mismatches, in which part of one short segment is partially complementary to part of a second; (d) and shifted correct matches, in which a misaligned overlap region is partially complementary to another region within the same overlap. Accordingly, in some embodiments, optimization comprises calculating a melting temperature for a single piece of desired DNA or DNA fragment, for example, for a hairpin. In some of these embodiments, the desired DNA or DNA fragment can be one that can be formed using at least one pair of primers. In some embodiments, optimization comprises calculating a melting temperature for a single piece of undesired DNA or DNA fragment. In some of these embodiments, the desired DNA or DNA fragment can be one that can be formed using at the same least one pair of primers. In some embodiments, optimization comprises calculating both types of melting temperatures.

The melting temperature gap is widened by perturbations to the codon assignments, including strengthening correct matches by increasing G-C content in the overlaps and disrupting incorrect matches by choosing non-complementary bases. Codon assignments are varied and the process repeated until the gap is comfortably wide. This process may be performed manually or automated. In a preferred embodiment, the search of possible codon assignments is mapped into an anytime branch and bound algorithm developed for biological applications, which is described in R. H. Lathrop et al. “Multi-Queue Branch-and-Bound Algorithm for Anytime Optimal Search with Biological Applications” in Proc. Intl. Conf. on Genome Informatics, Tokyo, Dec 17-19, 2001 pp. 73-82; in Genome Informatics 2001 (Genome Informatics Series No. 12), Universal Academy Press, the disclosure of which is incorporated by reference. Those skilled in the art will recognize that other optimization methods could be used, e.g., simulated annealing, genetic algorithms, other branch and bound techniques, hill-climbing, Monte Carlo methods, other search strategies, and the like. Those skilled in the art will further realize that optimizing, i.e., weakening, incorrect matches is functionally equivalent to optimizing, i.e., strengthening, correct matches, and vice versa. Consequently, suitable optimization methods include weakening incorrect matches, strengthening correct matches, and any combination thereof.

Those skilled in the art will realize that the size of the melting temperature gap is related to the annealing conditions such that a narrower gap may require more stringent annealing conditions in the assembly step to provide the requisite level of fidelity. Consequently, the temperature gap has no minimum value. In some embodiments, the temperature gap is greater than 0° C., at least about 1° C., at least about 2° C., at least about 3° C., at least about 4° C., at least about 5° C., at least about 6° C., at least about 7° C., at least about 8° C., at least about 9° C., at least about 10° C., at least about 12° C., at least about 14° C., at least about 16° C., at least about 18° C., or at least about 20° C. Those skilled in the art will understand that, under appropriate annealing conditions, the temperature gap is arbitrarily close to 0° C. Practically, the difference between the lowest-melting correct match and the highest melting incorrect match is at least about 1° C., more preferably, at least about 4° C., more preferably, at least about 8° C., most preferably, at least about 16° C. The wider the temperature gap, the more robust the self-assembly, thereby permitting the use of less stringent annealing conditions.

Those skilled in the art will appreciate that optimization may be performed using other parameters or measures related to hybridization propensity, for example, free energy, enthalpy, entropy, or other arithmetic or algebraic combinations of such parameters or measures, to achieve the same effect as melting temperature. Melting temperature itself is one such arithmetic or algebraic combination of such parameters or measures.

### Step 104: The Optimized Oligonucleotides are Obtained.

The optimized oligonucleotides are obtained, typically, for example, by synthetic preparation. In one embodiment, the oligonucleotides are single-stranded and overlapping portions of adjacent pieces are complementary. The optimized oligonucleotides can be designed such that the oligonucleotides are divided into two or more subsets. In one embodiment, for all oligonucleotides of a selected subset of the entire set of oligonucleotides, the lowest Tmc of any fully complementary IS/ISC pair is higher than the highest Tmi associated with any ISC within the subset. For example, all oligonucleotides of a first selected subset of the entire set, can be designed, where the result of the design is that the lowest Tmc of any fully complementary IS/ISC pair is higher than the highest Tmi associated with any ISC within the first subset, and all oligonucleotides of a second selected subset of the entire set, can be designed, where the result of the design is that the lowest Tmc of any fully complementary IS/ISC pair is higher than the highest Tmi associated with any ISC within the second subset. Codons of oligonucleotides may be selected from among synonymous codons using, for example, a computer optimization analysis. The oligonucleotides can be mutually and globally thermodynamically optimized by, for example, computer analysis as described in U.S. Patent Application 2005/0106590. In some such embodiments, the oligonucleotides of the first subset are non-overlapping. In some such embodiments, the oligonucleotides of the second subset are non-overlapping. In one embodiment, for all oligonucleotides of the entire set of oligonucleotides, the lowest Tmc of any fully complementary IS/ISC pair is higher than the highest Tmi associated with any ISC.

Step 105: Oligonucleotides Self-Assemble into First DNA Constructs.

The oligonucleotides are designed to self-assemble to form first DNA constructs either by a recursive assembly process or by a direct self-assembly process. In some embodiments, this oligonucleotide assembly is performed to form a group of polynucleotides. In other embodiments, the self-assembly process is not required and the set of non-assembled, or partially assembled oligonucleotides are used in further methods. Thus, the following description illustrates a method of assembly, extension and combination of oligonucleotides (e.g., steps 105-107 described herein) which is not required in all embodiments of the methods provided herein.

In a recursive assembly process, the oligonucleotides optimized for medium-sized pieces are combined to form medium-sized pieces, which are then combined to form first DNA constructs. The recursive assembly may include additional intermediate steps with, for example, pieces of additional intermediate sizes. In some embodiments, oligonucleotides self-assemble by a direct self-assembly process. In a direct self-assembly process, the optimized oligonucleotides are combined to form first DNA constructs.

Optimized oligonucleotides can self-assemble to form a DNA construct of single-stranded DNA (ssDNA) segments connected by double-stranded overlap regions. In embodiments in which the oligonucleotides are double-stranded, the pieces are preferably first denatured. Embodiments using overlap extension to reassemble a piece of DNA have single-stranded gaps between the double-stranded overlap regions. Preferably, the single-stranded gaps are from about zero to about 20 bases long. Embodiments using ligation to reassemble a piece of DNA have single-stranded gaps of length zero (i.e., no gap, a nick in the DNA) and the double-stranded overlap regions abut each other. Embodiments using cloning to reassemble a piece of DNA have any combination of gaps and no gaps.

### Step 106: DNA Constructs are Extended to One or More Optimized Polynucleotides.

DNA constructs can be extended to form one or more optimized polynucleotides. In embodiments with single-stranded gaps between the double-stranded overlaps, extension is accomplished using overlap extension, preferably, using a high-fidelity DNA polymerase reaction. In embodiments with no gaps between the double-stranded overlaps, extension is accomplished by ligation. In another embodiment, the self-assembled construct is cloned into an expression vector, and the extension to full-duplex double-stranded DNA (dsDNA) is performed by the cellular machinery.

Some embodiments use ssDNA in subsequent steps. ssDNA is produced from the dsDNA using any method known in the art, for example, by denaturing or using nicking enzymes. In some embodiments, the DNA is cloned into a vector that produces ssDNA, for example, bacteriophage M13 or a plasmid containing the M13 origin of DNA replication. M13 is known to roll-off ssDNA into the medium.

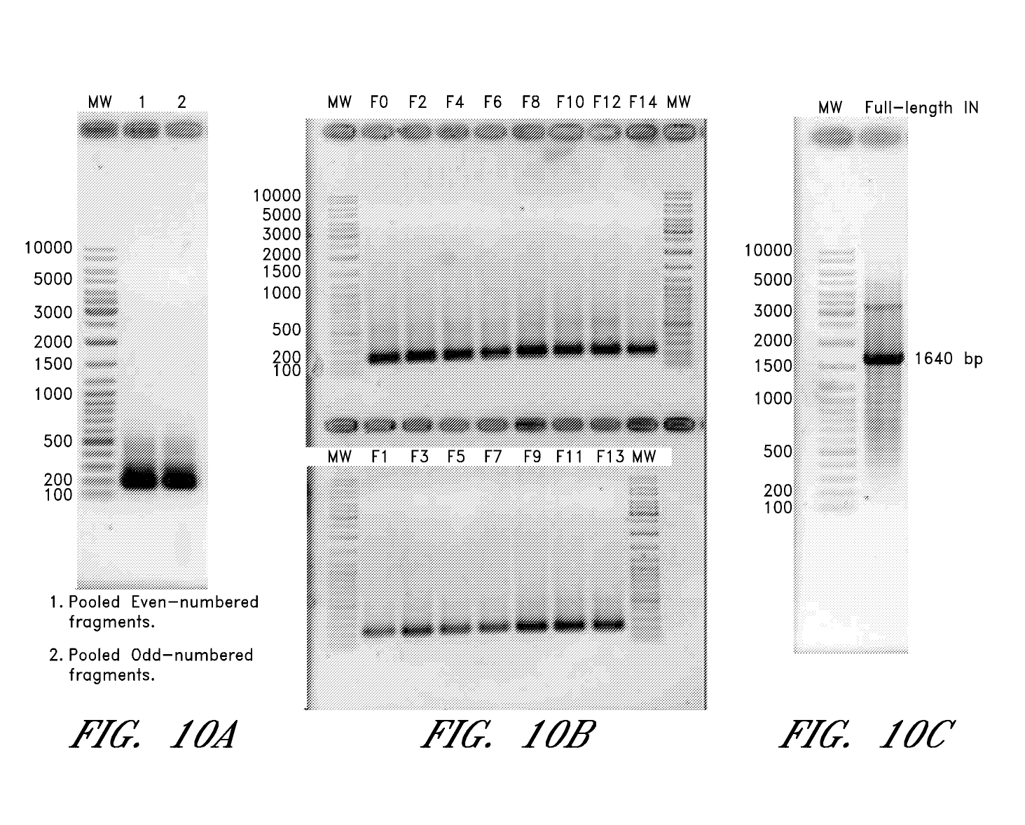

The optimized oligonucleotides can be designed, where the result of the design is that the oligonucleotides are divided into two or more subsets, where for all oligonucleotides of a selected subset of the entire set of oligonucleotides, the lowest Tmc of any fully complementary IS/ISC pair is higher than the highest Tmi associated with any ISC within the subset. In such an embodiment, two or more intermediate fragments can be assembled prior to assembling the full polynucleotides. An exemplary use of subsets can be seen by reference to FIG. 8. In one example, oligonucleotides are divided into two or more subsets, where a first subset contains the oligonucleotides making up fragments 1, 3, 5, 7, and 9 of FIG. 8, and a second subset contains the oligonucleotides making up fragments 2, 4, 6, 8, and 10 of FIG. 8. The oligonucleotides of the first subset can then be treated to self-assemble and be extended to form fragments 1, 3, 5, 7, and 9, while the oligonucleotides of the second subset can, in a separate reaction, be treated to self-assemble and be extended to form fragments 2, 4, 6, 8, and 10. Fragments 1-10 can then be combined, allowed to assemble, and extended to form the full-length polynucleotide. As can be readily appreciated, the lowest Tmc and highest Tmi for the first and second subsets need not be identical. One of skill in the art will recognize that such intermediate assembly of fragments and combination of fragments to form longer nucleic acid molecules can be performed in any of a variety of rounds and permutations. In some such embodiments, the oligonucleotides of the first subset are non-overlapping. In some such embodiments, the oligonucleotides of the second subset are non-overlapping.

In another embodiment, for all oligonucleotides of the entire set of oligonucleotides, the lowest Tmc of any fully complementary IS/ISC pair is higher than the highest Tmi associated with any ISC.

### Step 107: A Group of Optimized Polynucleotides is Combined.

A group of optimized polynucleotides is combined. In preferred embodiments, the group of optimized polynucleotides comprises polynucleotides formed in accordance with step 106. In some embodiments, the group of polynucleotides comprises optimized polynucleotides and further comprises un-optimized polynucleotides. In preferred embodiments, the group of optimized polynucleotides comprises polynucleotides containing the optimized oligonucleotides. Preferably all of the oligonucleotides of the plurality of optimized polynucleotides were globally and mutually optimized.

In some embodiments, the plurality of optimized polynucleotides comprises at least at least 2, 3, 4, 5, 6, 7, 8, 9, 10, 12, 15, 20, 25, 30, 35, 40, 50, 75, 100, 150 or more polynucleotides.

### Step 108: At Least One Primer is Provided.

The methods provided herein can utilize either a set of oligonucleotides, a set of partially assembled polynucleotides, a group of assembled polynucleotides, or combinations thereof in method of recombining nucleic acid sequences to form new recombined polynucleotides. The method of recombining two or more oligonucleotides or polynucleotides can take advantage of the fact the nucleic acid sequences to be recombined are uniquely thermodynamically addressable such that a first portion of a primer of sufficient length can be designed to preferentially hybridize to only a single first oligonucletide or polynucleotide while a second portion of a primer of sufficient length can be designed to preferentially hybridize to only a single second oligonucletide or polynucleotide. In some embodiments, a primer refers to a nucleic acid sequence that primes the synthesis of a polynucleotide in an amplification reaction. Typically a primer comprises fewer than about 150 nucleotides and may comprise fewer than about 30 nucleotides. Primers may range from about 8 to about 100 nucleotides. Use of such a primer can thus specifically combine the nucleotide sequences of the first oligonucletide or polynucleotide and the second oligonucletide or polynucleotide. The location of the primer-complementarity regions on the first and second oligonucleotides or polynucleotides is not limited because all sequences of the oligonucleotides or polynucleotides are uniquely thermodynamically addressable. Thus, a primer can be used to link the nucleotide sequence of a first oligonucleotide or polynucleotide and a second oligonucleotide such that regions of the encoded polypeptide are combined, to form, for example, a “shuffled” polynucleotide, a chimeric polypeptide, a polypeptide containing mutations, deletions or insertions. Such recombined polynucleotides can encode a “shuffled” polypeptide, a fusion polypeptide, a polypeptide containing mutations, deletions or insertions. Any of a variety of primer designs are possible in accordance with the methods provided herein and the knowledge in the art.

The group of polynucleotides can be selected according to the desired variability of sequences to be available for recombination. For example, a first subgroup of the group of polynucleotides can share a higher sequence identity than other members of the group of polynucleotides. Recombination of sequences within such a subgroup can result in shuffled or chimeric polynucleotides that differ only slightly within the recombined region. In one instance, the slight variation may be a nucleotide change that are expected to result in little or no change in the secondary or tertiary structure of the protein, or result in little or no change in ligand binding, protein binding, or enzymatic activity. In another example, the group of polynucleotides can contain widely varying sequences that, when recombined, are expected to result in more drastic changes in secondary or tertiary structure of the protein, or result in ligand binding, protein binding, or enzymatic activity.

Sufficient length of a portion of a primer to preferentially hybridize to an oligonucletide or polynucleotide can be at least 3, 4, 5, 6, 7, 8, 9, 10, 11, 12, 13, 14, 15, 16, 17, 18, 19, 20, 22, 24, 26, 28, 30, 32, 36, 38, 40, 50, 60, 70, 80, 90 or 100 nucleotides. A primer may be between about 6 and about 250 bases. Typically, a primer will be at least, or at least about, 6, 7, 8, 9, 10, 11, 12, 13, 14, 15, 16, 17, 18, 19, 20, 22, 24, 26, 28, 30, 32, 36, 38, 40, 45, 50, 55, 60, 65, 70, 75, 80, 85, 90, 95, 100, 110, 120, 130, 140, 150, 160, 170, 180, 190 or 200 bases in length. Often, a primer will be no more than, or no more than about 10, 11, 12, 13, 14, 15, 16, 17, 18, 19, 20, 22, 24, 26, 28, 30, 32, 36, 38, 40, 45, 50, 55, 60, 65, 70, 75, 80, 85, 90, 95, 100, 110, 120, 130, 140, 150, 160, 170, 180, 190, 200, 225 or 250 bases in length The sufficient length can be a function of several factors including the Tmc and Tmi values, complexity of the set of oligonucletides or group of polynucleotides, and any specific characteristics of the set of oligonucletides or group of polynucleotides. Such factors will be readily apparent to one skilled in the art for a particular set of oligonucletides or group of polynucleotides, and the desired length of a preferentially hybridizing portion of a primer can be determined in accordance with the teachings provided herein or otherwise known in the art. A primer property, such as the length of a primer or the length of a portion of the primer, can be designed to ensure in-frame coding between the recombined sequences. That is, a primer can be designed such that the 3-base codon repeat of the first oligonucleotide or polynucleotide is in-frame with the 3-base codon repeat of the second oligonucleotide or polynucleotide. When the coding regions of the polynucleotides are known, primers designed to ensure in-frame coding can be readily designed in accordance with the teachings provided herein or otherwise known in the art.

Thus, in one embodiment, a primer is provided that has a first region that is fully complementary to a sequence S1 of a first oligonucleotide or polynucleotide, wherein sequence S1 is of a minimum length of about 5 bases and has a second region that is fully complementary to a sequence S2 of a second oligonucleotide or polynucleotide, wherein sequence S2 is of a minimum length of about 5 bases. The lengths of S1 and S2 may be, for example, at least about 6 bases, at least about 7 bases, at least about 8 bases, at least about 9 bases, at least about 10 bases, or at least about 15 bases. The lengths of S1 and S2 may be, for example, no more than about 20 bases, no more than about 18 bases, no more than about 15 bases, no more than about 14 bases, no more than about 13 bases, no more than about 12 bases, no more than about 11 base, or no more than about 10 bases. In some embodiments, the lengths of S1 and S2 are between about 9 and about 13 bases. In such primers, codons of the oligonucleotides of the oligonucleotide set can be selected from among synonymous codons, and as a result, the melting temperature of the hybridization of the first region of the first primer to S1 is greater than the melting temperature of any incorrect hybridization of the first region to any other sequence of the set and the melting temperature of the hybridization of the second region of the second primer to S2 is greater than the melting temperature of any incorrect hybridization of the second region to any other sequences in the set.

Also contemplated herein is the use of primer pairs containing complementary, or at least overlapping, nucleotide sequences in both a first region complementary to a first oligonucleotide or polynucleotide and a second region complementary to a second oligonucleotide or polynucleotide. That is, a first primer of a primer pair can have a first region that is fully complementary to a sequence S1 of a first oligonucleotide or polynucleotide, and a second region that is fully complementary to a sequence S2 of a second oligonucleotide or polynucleotide, and a second primer of the primer pair can have third and fourth regions respectively complementary to the first and second regions of the first primer. For example, in one embodiment at least one pair of internal primers is provided and can be combined with the set of oligonucleotides that are not assembled, partially assembled, or fully assembled in to a group of optimized polynucleotides. All of the at least one pair of internal primers can simultaneously contact the set of oligonucleotides or group of polynucleotides, or the set of oligonucleotides or group of polynucleotides can be serially contacted by different pairs of primers. In some instances, a primer pair can be used to introduce a sequence not in a set of oligonucleotides. Two or more primer pairs may be used, for example, to introduce a plurality of sequence substitutions.

Primers and/or primer pairs can be used to control any of a variety of factors in polynucleotide recombination. For example, the primer and/or primer pairs can be used to control the number of incorporated sequences. In one instance, the sequences of the primers and/or primer pairs can dictate the regions of sequences to be incorporated. In instances in which a set of oligonucleotides is globally and mutually thermodynamically optimized, a primer and/or primer pair sequence can restrict the number of incorporations per polynucleotide to a fixed number. In some instances, the relative amount of the primer and/or primer pairs to oligonucleotides and/or polynucleotides can be used to at least partially control the frequency of recombination of the sequences. For example, a smaller concentration of primers and/or primer pairs can lead to less sequence incorporations than a larger concentration. In some instances, the timing of addition of the primer or/primer pair can be used to at least partially control the frequency of recombination of the sequences. For example, a first primer pair can be added prior to the first extension cycle, and a second primer pair can be added subsequent to multiple cycles (e.g., subsequent to 2, 4, 6, 8, or 10 cycles) to thereby differentially control the rate of recombination of the sequences linked by the primer pairs.

Primers and/or primer pairs can be used to control sites of incorporations. In one instance, the sequences of the primers and/or primer pairs can dictate where sequences will be incorporated. In instances in which a set of oligonucleotides is globally and mutually thermodynamically optimized, a primer and/or primer pair sequence can direct the sites of incorporations per polynucleotide to fixed locations.

Primers and/or primer pairs can be used to control combinations of sequence incorporations. In one instance, the sequences of the primers and/or primer pairs may dictate which sequences can be incorporated after incorporation of an initial sequence. For example, a primer pair can be used to incorporate additional sequences into a polynucleotide thus permitting a substitution or insertion that otherwise could not occur or prevent a substitution that otherwise could occur.

In some embodiments, the at least one pair of internal primers is a plurality of primers. The first and second primer of an internal primer pair may be designed such that the a first region of the first primer is fully complementary to a given sequence S1 of a first polynucleotide of a polynucleotide group, a second region of the second primer is fully complementary to a sequence S2 of a second polynucleotide of the polynucleotide group, and a third region of the first primer is identical to, or fully complementary to, a fourth region of the second primer. These primers may be referred to as reshuffling primers. In this instance, the first and second primer of an internal primer pair may be designed such that a first region of the first primer is uniquely complementary to a given first nucleotide internal sequence, a second region of the second primer is uniquely complementary to a specific second nucleotide internal sequence, and the third and fourth regions of the first and second primers, respectively, are identical to or uniquely complementary to each other. By such a configuration, the complementarity or identity between the third and fourth regions can serve to link the sequence of the first oligonucleotide with the sequence of the second oligonucleotide, and in so doing, can permit point or block mutations and/or insertions not otherwise available by directly linking the sequences of the first and second oligonucleotides.

In some such embodiments, the third region of the first primer comprises all or a portion of the first region of the first primer and the fourth region of the second primer comprises all or a portion of the second region of the second primer. In such instances, the sequence of the first primer that is identical to or complementary to the sequence of the second primer also is complementary to or identical to all or a portion of S2. Similarly, the sequence of the second primer that is identical to or complementary to the sequence of the first primer also is complementary to or identical to all or a portion of S1. Often there will be sequence overlap between the first and second primers to facilitate primer extension formation of the entire intended chimeric polypeptide. This sequence overlap in some instances includes the portions of the primers that are complementary to polynucleotide sequences. In some such embodiments, the third region of the first primer consists of all or a portion of the first region of the first primer and the fourth region of the second primer consists of all or a portion of the second region of the second primer. Thus, for some first and second primers, these share sequences identical to or complementary to each other and to S1 and/or S2 and do not insert additional nucleotides or mutate portions of either polynucleotide being combined. In other embodiments, the third region of the first primer comprises one or more bases not identical to or not complementary to S2 and the fourth region of the second primer comprises one or more bases not identical to or not complementary to S1. Thus, for some first and second primers, these share sequences identical to or complementary to each other and to S1 and/or S2 and insert additional nucleotides between polynucleotides being combined or mutate portions of either or both polynucleotide being combined.

Codons of the oligonucleotides of the oligonucleotide set may be selected from among synonymous codons, and as a result, the melting temperature of the hybridization of the first region of the first primer to S1 is greater than the melting temperature of any incorrect hybridization of the first region to any other sequence of the set and the melting temperature of the hybridization of the second region of the second primer to S2 is greater than the melting temperature of any incorrect hybridization of the second region to any other sequences in the set. The lengths of S1 and S2 can be, for example, at least about 6 bases, at least about 7 bases, at least about 8 bases, at least about 9 bases, at least about 10 bases, or at least about 15 bases. The lengths of S1 and S2 can be, for example, no more than about 20 bases, no more than about 18 bases, no more than about 15 bases, no more than about 14 bases, no more than about 13 bases, no more than about 12 bases, no more than about 11 base, or no more than about 10 bases. In some embodiments, the lengths of S1 and S2 are between about 9 and about 13 bases.

A primer as provided herein can also contain an optional third region that is not required to be complementary to a oligonucleotide or polynucleotide region or to another primer region. For example, a primer can contain a nucleotide sequence that encodes a mutation not provided in any oligonucleotide or polynucleotide region. In another example, a primer can contain a nucleotide sequence that encodes an insertion of a sequence not provided in any oligonucleotide or polynucleotide region. Further, in some embodiments, the region of complementarity between primer pairs can contain a nucleotide sequences that encodes a mutation or insertion of a sequence not provided in any oligonucleotide or polynucleotide region.

In some embodiments, plurality of different primers can be provided. Each of the plurality of primers can have a first region uniquely complementary to a sequence of one polynucleotide of a group of polynucleotides and a second region uniquely complementary to a sequence of another polynucleotide of the group, and, optionally, a third region not complementary to any polynucleotides of the group. The plurality of primers can differ from each other in at least the first region, the second region, or the third region. In some embodiments, at least some of the plurality of primers are identical to or complementary to each other in one, two or three of the first, second and third regions. The primers of the plurality of primers may be the same length or of different lengths. In some embodiments, the number of primers provided is greater than the number of polynucleotides in the group. In some embodiments, the number of primers provided is less than the number of polynucleotides in the group. In some embodiments, the number of primers provided is about equal to the number of polynucleotides in the group.

Primer characteristics may be determined by the type of desired mutation. The mutation may be, for example, a point mutation, as illustrated in FIG. 4A, a regional mutation, as illustrated in FIG. 4B, or a directed reshuffling mutation, as illustrated in FIG. 4C. Methods of primer design in order to accomplish such mutations are readily available to those of skill in the art in accordance with the teachings provided herein.

The lengths of the primers can depend on the number of optimized polynucleotides combined in step 107 and the sequences thereof. The lengths of the primers can depend on the number of oligonucleotides optimized in step 103 and sequences thereof. For example, to ensure that a primer is uniquely complementary to a given internal sequence, the primer length may be longer when the number of combined polynucleotides is greater or when the sequence homology between the combined polynucleotides is greater. In some embodiments, the primer length is less than the length of the optimized oligonucleotides. The length of a primer can depend, for example, on an annealing temperature and/or a melting temperature. For example, longer primers can use higher annealing temperatures and thereby specifically target sequences for recombination. The lengths of the primers may be, for example, greater than 3, 4, 5, 6, 7, 8, 9, 10, 11, 12, 13, 14, 15, 16, 17, 18, 19, 20, 22, 24, 26, 28, 30, 32, 36, 38, 40, 50, 60, 70, 80, 90, 100, 110, 120, 140 or 150 bases. In some preferred embodiments, the lengths of the primers are greater than about 10 bases. In some more preferred embodiments, the lengths of the primers are greater than about 15 bases. In some most preferred embodiments, the lengths of the primers are about 22 bases. In some embodiments, the lengths of the primers are less than about 150, 140, 120, 110, 100, 90, 80, 70, 60, 50, 40, 38, 36, 34, 32, 30, 28, 26, 24, 22, 20, 19, 18, 17, 16, 15, 14, 13, 12, 11, 10, 9, 8, 7, 6 or 5 bases long. In some preferred embodiments, the lengths of the primers are between about 18 and about 25 bases long.

The GC-content of the primers, for example, may be optimized. In some embodiments, the GC-content is between about 40% and about 60%.

In some embodiments, primers are designed to form DNA sequences containing sequences of multiple polynucleotides. Sequences of primers can be designed to be uniquely complementary to sequences of the polynucleotides. A primer sequence may be chosen based on the unique thermodynamic address of the sequence with which it is desirable for the primer to hybridize. In some embodiments, primers allow for specific point mutations to be formed in DNA sequences. In some embodiments, the codons of the polynucleotides of the set have been selected from among synonymous codons, and as a result, for the melting temperature of the hybridization of a specific region of a specific primer is higher for a sequence S of the polynucleotide than for any other sequence in the group of polynucleotides.

In some embodiments, a large amount of primers are provided relative to the amount of optimized polynucleotides. The large amount of primers can facilitate a higher rate of recombination brought about by incorporation of the primers. In one instance, the probability that a given substitution will occur increases with the large amount of primers. In another instance, the probability that multiple different substitutions will occur within one or across many polynucleotides or oligonucleotides increases with the large amount of multiple primers. In some embodiments, a large amount of primers with the same sequence are provided relative to the amount of optimized polynucleotides to which the primers are complementary. Thus, the probability that a polynucleotide or oligonucleotide will be substituted by a desired sequence can increase due to the large relative concentration of primers. In some embodiments, a variety of primer pairs in provided. Therefore, a plurality of substitutions within an individual polynucleotide or oligonucleotide or across a plurality of polynucleotides or oligonucleotides can be expected.

External primers can also be provided. The number of external primers may be less than the number of internal primers provided. The external primers may be complementary to the end sequences of optimized polynucleotides. In some embodiments, the external primers can be truncations of the optimized polynucleotides. For example, an external primer can be complementary to all but the 1, 2, 3, 4, 5, 6, 7, 8, 9, 10, 12, 15, 20, 25, or 30 most N-terminal codons of a polynucleotide. In another example, an external primer can be complementary to all but the 1, 2, 3, 4, 5, 6, 7, 8, 9, 10, 12, 15, 20, 25, or 30 most C-terminal codons of a polynucleotide. Use of truncated external primers can thus be used to provide additional diversity to the number of recombined sequences generated in accordance with the methods provided herein.

In some embodiments, primers are provided such that each of the primers is simultaneously contacted with assembled polynucleotides of the group. In some embodiments, primers are provided such that the group of polynucleotides is serially contacted with different pluralities of primers. In some embodiments, primers are provided such that each of the primers is simultaneously contacted with assembled oligonucleotides of the set. In some embodiments, primers are provided such that the set of oligonucleotides is serially contacted with different pluralities of primers. Embodiments including serially contacting oligonucleotides or polynucleotides can be used, for example, to provide specific concentrations of specific DNA. For example, if a set of first primers are first contacted with a set of oligonucleotides or group of polynucleotides and second primers subsequently hybridize with the extended products thereof, the final resultant product can contain sequences associated with the first primers in a higher concentration than sequences associated with the second primers.

### Step 109: Primer Hybridizes to Optimized Oligonucleotides or Polynucleotides and Optionally to Complementary Primers to Form a Primer-Oligonucleotide or Primer-Polynucleotide Duplex.

The primer is allowed to hybridize to the corresponding complementary sequence of the optimized polynucleotide or oligonucleotide. In some embodiments, for example in which directed reshuffling of polynucleotides is desired, primers may additionally hybridize to other complementary primers. In other embodiments, primers do not hybridize to other primers. For examples a first primer can be complementary to a first region of a first polynucleotide and a second region of a second polynucleotide and a second primer can be complementary to the complement of a third region of the second polynucleotide and a fourth region of a third polynucleotide. Typically, extension of such primers is performed in the presence of the first polynucleotide, the second polynucleotide and a polynucleotide at least partially complementary to the second polynucleotide. A similar method can be performed using corresponding oligonucleotides.

One or more primers may be combined with oligonucleotides or polynucleotides. This combination can, for example, allow for hybridization of complementary sequences thereby producing primer-oligonucleotide or primer-polynucleotide duplexes. In some embodiments, the concentration of a primer is greater than, at least 2 times greater than, at least 3 times greater than, at least 4 times greater than, at least 5 times greater than or at least 10 times greater than the concentration of an oligonucleotide or polynucleotide of any given sequence with which the primer is combined.

One or more primers can be contacted with an oligonucleotide or a polynucleotide or a set thereof comprising a region complementary to a region of the primer. A set of oligonucleotides or a group of polynucleotides can include at least three different oligonucleotides or polynucleotides, respectively. Each primer can be simultaneously contacted with a group of assembled polynucleotides. In some embodiments, a set of oligonucleotides is serially contacted with different primers. The contacting can, for example, allow for hybridization of complementary sequences thereby producing primer-oligonucleotide or primer-polynucleotide duplexes.

In some embodiments, a resultant primer-oligonucleotide or primer-polynucleotide hybridization is PCR amplified to create a chimeric polynucleotide having some sequence from a first polynucleotide and some sequence from a second polynucleotide.

In some embodiments, a single primer can hybridize with one of a plurality of different polynucleotides. For example, two polynucleotides may have different first sequences but the same second sequences. When a primer is configured to hybridize with all or part of the second sequence, it can hybridize with either of the two polynucleotides. Such primer design can be used, for example, to increase the diversity of the recombined sequences generated relative to the number of primers used to generate such recombined sequences.

In step 103, oligonucleotides can be optimized such that a first melting temperature of a correct hybridization between a given internal nucleotide sequence and complementary nucleotide sequence can be higher, and in some embodiments substantially higher, than a second melting temperature of a incorrect hybridization between that same given internal nucleotide sequence and any other portion of the group of polynucleotides. As described above, this optimization can produce a large gap between melting temperatures of correct hybridizations and incorrect hybridizations. Because a primer can be designed to have complementary sequences to a specific internal sequence of an optimized polynucleotide, the primer also can be designed to have a first melting temperature for the correct hybridization to the specific internal sequence that is higher, and in some embodiments substantially higher, than the second melting temperature for the incorrect hybridization to any other internal sequence. Therefore, the primers can be particularly likely to hybridize with the desired internal sequences, and thus, can be designed to specifically target any portion of an optimized polynucleotide.

### Step 110: the Primer-Oligonucleotide or Primer Polynucleotide Duplexes are Extended to a Resulting Full-Duplex DNA.

The primer-oligonucleotide or primer-polynucleotide duplexes can be extended to form a resulting full-duplex DNA. In some embodiments, multiple full-duplex DNA sequences are formed. In some embodiments, the full-duplex DNA sequences are the sequences of a gene, which may be a mutant gene or a chimeric gene. In some embodiments, the resulting full-duplex DNA sequences are DNA fragments. The DNA fragments may later be combined with other DNA fragments to form longer DNA sequences, which may be DNA sequences of a gene.

Extension is preferably accomplished using overlap extension, preferably, using a high-fidelity DNA polymerase reaction, though it may be accomplished by other methods known in the art.

Some embodiments use ssDNA in subsequent steps. ssDNA is produced from the dsDNA using any method known in the art, for example, by denaturing or using nicking enzymes. In some embodiments, the DNA is cloned into a vector that produces ssDNA, for example, bacteriophage M 13 or a plasmid containing the M13 origin of DNA replication. M13 is known to roll-off ssDNA into the medium.

### Step 111: A Property Indicative of the Resulting Full-Duplex DNA is Determined.

A selection or screen can be used to identify the resulting DNA products. For example, a selection or screen can be used to confirm one or more desired properties of the resulting DNA products, including, but not limited to, preservation of polypeptide encoding frame or length of polypeptide encoding sequence. In some embodiments, a synthetic gene comprising a DNA piece is fully reassembled and a resultant polypeptide is then screened or selected for a desired property. For example, a polypeptide associated with the resulting full-duplex DNA may be analyzed by gel electrophoresis, capillary electrophoresis, two-dimensional electrophoresis, isoelectric focusing, spectroscopy, mass spectroscopy, NMR spectroscopy, chemically, ligand binding, enzymatic cleavage, and/or a functional or immunological assay. Based on the results of the screening or selection, nucleotide sequences can be identified that are associated with a polypeptide having a desired property. In one example, electrophoresis can identify if the polypeptide has a correct or incorrect weight as compared to, for example, other polypeptides or a fixed weight. In one instance, DNA sequences can be transferred into selected organisms such that a clone containing a correct and/or likely-to-be correct DNA sequence will exhibit a phenotype different from the phenotype exhibited by a clone containing an incorrect DNA sequence. In some instances, a “frame-shifted” vector is used as, for example, described herein or otherwise known in the art, to identify DNA having the desired sequences. Full-duplex DNA passing the selection, screening or analysis is typically further replicated and harvested.

Steps of the described method 100 may be combined, reordered, or eliminated. For example, steps 107 and 108 may be combined, steps 106 and 107 may be reordered, and/or step 111 may be eliminated. Steps 105 to 107 also may be eliminated. Additional steps may be added to the described method 100. It will be understood that such combining, re-ordering, eliminating, and/or adding of steps may slightly modify the steps. For example, if steps 106 and steps 107 are reordered, then the steps would then comprise combining a plurality of DNA constructs (instead of optimized polynucleotides) and extending the combined DNA constructs.

In a preferred embodiment, a disclosed method takes advantage of the fact that the genetic code is sufficiently degenerate to allow codons to be assigned so that, with high probability, wrong hybridizations melt at lower temperatures and correct hybridizations melt at higher temperatures. Consequently, there is an intermediate temperature range within which, with high probability, the product that does form is mostly correct. Because errors occur with low probability, two or more compensating errors that yield a product with the correct molecular weight—i.e., the same band in the final gel—or two or more compensating deletions that yield a product of the same reading frame—i.e., the same or nearly the same encoded amino acid sequence—would correspond to a doubly rare or rarer event.

### Recombination

The compositions and methods provided herein can be used to rationally and deliberately recombine nucleotide sequences of various polynucleotides, and find particular applications in methods directed to generating new polypeptide-encoding nucleotide sequences. If desired, the methods provided herein also can be used to arbitrarily recombine nucleotide sequences of various polynucleotides to arrive at new sequences that can be screened for desired properties, such as the ability to encode a polypeptide having desired properties. The recombination methods directed to generating new polypeptide-encoding nucleotide sequences can be directed to any of a variety of applications for generating new polypeptides, including, but not limited to, shuffling methods, N-terminus and C-terminus truncation methods for applications such as protein crystallization, solubility optimization methods, polypeptide structure/function relationship analysis, molecular evolution, improved design of polypeptides to have particularly desired properties such as pharmaceutical or industrial properties, methods of creating insertions and/or mutations and/or deletions in a polypeptide that would otherwise be laborious and time consuming.

### Gene Shuffling

In some embodiments, methods disclosed herein can be used as or in combination with gene-shuffling methods. In vitro molecular evolution can attempt to produce a resulting gene from an initial gene, such that the protein coded by the resulting gene is characterized by a specific property. A library of genes with a variety of mutations can first be generated from the initial gene. Techniques used to generate the library of genes can include whole genome mutagenesis, random cassette mutagenesis, error-prone PCR, and DNA shuffling, as known in the art. Additional methods include partial digestion of related genes, coupled with low-stringency hybridization and primer extension methods and/or ligation methods, as known in the art. Methods disclosed herein may be used instead of or in addition to these techniques to generate the library. Methods disclosed herein may provide simple generation strategies of producing large and/or diverse genetic libraries. The genes can then be screened to determine if they are associated with the desired property. Any of a number of screening methods known in the art, including, for example, phage display methods, can be used. In some embodiments, genes can then be selected to determine if they are associated with the desired property. Any of a variety of selection methods known in the art, including, for example, antibiotic resistance, can be used. Genes associated with the desired property can be separated, and specific nucleic acid sequences of the separated genes can be identified. In some instances, it may be advantageous to generate additional genes which are a combination of the separated genes.

Oligonucleotides of the separated genes or of DNA fragments of the separated genes can be optimized as described above to be, for example, uniquely thermodynamically addressable. Primers can then be provided in order to combine various regions of different genes or gene fragments. The plurality of initial un-optimized polynucleotides provided in step 101 of method 100, the plurality of polynucleotides containing the oligonucleotides optimized in step 103 of method 100, and/or the plurality of optimized polynucleotides combined in step 107 of method 100 can each encode at least a portion of a protein from the same protein family or superfamily. A protein family comprises a number of evolutionarily related proteins, and a superfamily comprises a number of related families.

### Compositions

In some embodiments, the present invention relates to a composition comprising a set of oligonucleotides which have been optimized as described above, such as to, for example, achieve a DNA melting temperature gap between correct (high melting temperature) and incorrect (low melting temperature) hybridizations and that can assemble to form polynucleotides. The set of oligonucleotides can comprise at least 2, 3, 4, 5, 10, 15, 20, 30, 40, 50, 60, 70, 80, 90, 100, 125, 150, 175, 200, 250 or 300 oligonucleotides that can assemble to form at least 2, 3, 4, 5, 10, 15 or 20 polynucleotides.

In some embodiments, the present invention relates to a composition comprising a group of polynucleotides, wherein sequences in the component oligonucleotides of the polynucleotides of the group have been optimized as described above, such as to, for example, achieve a DNA melting temperature gap between correct (high melting temperature) and incorrect (low melting temperature) hybridizations. The group of polynucleotides can comprise at least 2, 3, 4, 5, 10, 15 or 20 polynucleotides.

In some embodiments, the present invention relates to a composition comprising a set of partially assembled oligonucleotides, wherein sequences in the component oligonucleotides have been optimized as described above, such as to, for example, achieve a DNA melting temperature gap between correct (high melting temperature) and incorrect (low melting temperature) hybridizations. The set of partially assembled oligonucleotides can comprise at least 2, 3, 4, 5, 10, 15, 20, 30, 40, 50, 60, 70, 80, 90, 100, 125, 150, 175, 200 or 250 partially assembled oligonucleotides that ultimately assemble to form at least 2, 3, 4, 5, 10, 15 or 20 polynucleotides.

In some embodiments, the composition may further comprise at least one primer or pair of primers, as were described above. In some embodiments, each primer is about 5, 10, 15, 20, or 25 bases long. In some embodiments, each primer is between about 18 and about 25 bases long or about 9 and about 13 bases long. Compositions may further comprise a component that can be used to identify defective sequences or to identify non-defective sequences, which can be, for example, used to identify defective optimized oligonucleotides, optimized polynucleotides or resulting DNA sequences. Such a component can be, for example, an expression system used to select or screen for properly assembled polynucleotides, a gel electrophoresis system for evaluating the size of assembled polynucleotides, and other components known in the art for evaluating polynucleotide characteristics. The identifying component can include, for example, nucleic acid constructs and/or instructions for performing a screen or selection as disclosed herein. The identifying component can include, for example, gel electrophoresis, capillary electrophoresis, two-dimensional electrophoresis, isoelectric focusing, spectroscopy, mass spectroscopy, NMR spectroscopy, chemically, ligand binding, enzymatic cleavage, and/or a functional or immunological assay. The identifying component can compare molecular weights of a polypeptide. The identifying component can include a reagent configured to transform a DNA sequence into a selected organism such that a clone containing a correct and/or likely-to-be correct DNA sequence will exhibit a phenotype different from the phenotype exhibited by a clone containing an incorrect DNA sequence. The identifying and/or selecting means may include a “frameshifted” vector, as described herein or otherwise known in the art. The identifying component can include an expression vector or a cell-free expression system for expressing a polypeptide from a sample from a population of synthetic DNA sequences.

In some embodiments, the present invention relates to a composition comprising a set or plurality of oligonucleotides and/or polynucleotides as described herein along with the documentation of the sequences of the set or plurality of oligonucleotides and/or polynucleotides.

### Software

In some embodiments, the present invention relates to a computer-readable medium having software modules for: receiving sequences of a plurality of polynucleotides; calculating optimized synonymous sequences that are uniquely thermodynamically accessible; and outputting the optimized sequences. Software modules may also provide the ability to stop, save, and restart user sessions at a later time; validate and/or error check the input sequences and receive batch input, which, for example, may be imported from other files. Software modules may also provide users with options such as, for example, the set of polynucleotides to be optimized. Computer-readable media can contain sequences of optimized polynucleotides, optimized oligonucleotides or partially assembled optimized oligonucleotides. Computer readable media can contain the sequences of primers to be contacted with the set of oligonucleotides, set of partially assembled oligonucleotides or group of polynucleotides alone, or in combination with sequences of optimized polynucleotides, optimized oligonucleotides or partially assembled optimized oligonucleotides.

### Thermodynamic System Improvements

The Thermodynamics of Structures Likely to Occur Just Before Final Melting are of interest to gene self-assembly. High melting temperature (Tm) structures are those most likely to persist, even transiently, at high PCR temperatures, and may interfere with correct hybridization and extension to a single PCR product. Thermodynamics and optimization seek to eliminate such structure. However, identifying the highest Tm structures of a given sequence is difficult. The RNA folding problem is conjectured to be NP-hard because it shares many characteristics with protein folding, which is known to be NP-hard.

Software modules can compute thermodynamic output from energy data, a sequence, and a list of constraints. Software modules can be improved by eliminating intermediate file output to the Network File System, post-processing to compute Tms, graphics and interactive features, additional constraints, searches for multi-branch loops, low Tm structure retention, and certain inefficiencies in the choice of base pairs for beginning tracebacks.

### Sequence Search Improvements

Software modules may enable “energy parameters” to be constrained in knowledge-directed ways, which can succeed in forcing the prediction of structures containing, for example, a “small number” of helices connected by “not large” and “not very asymmetric” interior loops. Such parameters can reduce the time of the calculations.

Constraint satisfaction, an NP-hard problem, can correspond to the need to avoid undesired high-Tm secondary structure, prohibited patterns, rare codons, RNA splice sites, promotes, control signals, and other undesired sequence properties, within the gene and its assembly. The graph articulation point protein side-chain rotamer selection algorithm of Canutescu A. A., et al. (2003) Protein Sci, 12, 2001-2014, which is herein incorporated by reference in its entirety, can be included into software described herein. It includes the idea that articulation points in the constraint graph correspond to control points in the search, because they factor the solution graph cleanly into two conditional sub-problems given the value assumed by the articulation point. In sequence optimization, an articulation point might be a high-Tin secondary structure that connected two otherwise disjoint webs of high-Tm secondary structure. This can provide an efficient way to list the interacting secondary structure positions, and a search subsystem can use this list to make more efficient sequence search choices given the secondary structures known to be present. This can lead to technical improvements including better tracking of mfold helices; the ability to retrieve multiple mfold folds from a single sequence run; cluster load balancing; improved pattern extraction from mfold; extensive caching and pre-processing; user control of run parameters; user control of prohibited regular patterns; automatic check-pointing, error-handling and restarts; and a queue key.

Identification of Incorrect Hybridization Events to be Removed from Calculations

The highest Tin structures are the ones most likely to persist, even transiently, at the elevated temperatures of primer extension reactions. If present, these structures may interfere with correct hybridization and extension to a single PCR product. However, it is difficult to identify these structures.

Different possible codon substitutions at each amino acid position of the sequence being designed lead to different possible bases at each base position. Such degenerate base positions may be represented compactly by an IUPAC convention, where A, C, G, and T represent themselves, and M=AC; R=AG, W=AT; S=CG; Y=CT; K=GT; V=ACG; H=ACT; D=AGT; B=CGT; and N=ACGT. A string of IUPAC codes can represent compactly all possible base substitutions of the sequence, fragments, and oligonucleotides in the design. The highest Tin folding of such an IUPAC string, across al possible base replacements at each degenerate base position, would yield an upper bound on Tm for possible foldings under any set of codon substitutions. This upper bound would allow a pre-processing step to prune certain potential incorrection hybridization events that could never result in a Tm high enough to affect sequence design and optimization. Thus, thermodynamics calculations by a software module concerning these potential hybridization events would never need to be done again.

### Refolding

If a sequence is refolded thermodynamically at the Tm predicted by a first folding, using the same ion concentrations and the original constraints, the resulting free energy ΔGₘᵢₙ can provide valuable information. By definition, the computed highest Tm structure will have a free energy of 0 at the predicted ΔGₘᵢₙ and a free energy greater than 0 for any temperature above that Tm. If refolding shows that ΔGₘᵢₙ is greater than or equal to 0, then no stable structure exists at that temperature, meaning that the highest Tm structure has been found. Only if ΔGₘᵢₙ<0, which is very rare, will an alternative structure with a higher melting temperature exist and require further analysis. Thus, for the vast majority of oligonucleotide pairs, refolding can eliminate the need for iterative search and further testing to find the highest Tm structure.

After each sequence is first folded, auxiliary arrays can be filled in with free energy parameters for refolding at the predicted Tm. Both enthalpies and free energies at T_{Asmbl} can be stored, and the mono and divalent ion effects can already be encoded in the known free energies. Free energies at temperature T are given by:

$$Δ {G}_{T} = \frac{T}{{T}_{Asmbl}} \times Δ {G}_{Asmbl} + ( 1 - \frac{T}{{T}_{Asmbl}} ) \times Δ H$$

where ΔG_{Asmbl} refers to a free energy at T. No structure prediction is needed, only a minimum free energy prediction, which can reduce refolding time by over a factor of two.

In some embodiments, all sequence optimizations can be hard-wired by a system architecture to produce a single linear gene assembly.

### Multiple Tracebacks

In some embodiments, a software module can use a recursive fill algorithm to fill arrays, followed by tracebacks which generate structures by tracing paths through the filled arrays. A traceback strategy dictates the choices made along the path. The use of different traceback strategies can generate more diverse structures and increase the likelihood of finding the highest Tm structure. In other embodiments, a software module can perform a time consuming fill algorithm more than once using different parameter settings, followed in each case by tracebacks to generate structures.

### Sequences Composed of IUPAC Degenerate Base Codes

A software module may determine oligonucleotides and oligonucleotide pairs that could not form unwanted structures at TAsmbl, no matter which codon choices were made in sequence design. The algorithm may produce the worst possible scenario by predicting the lowest free energy that could result from synonymous base substitutions. Careful analysis of the energy parameters established that: (1) a C:G or G:C base pair always leads to a lower free energy for any motif containing it than do the other four possibilities; (2) an A:T or T:A base pair is always preferable to a G:T or T:G wobble pair; and (3) a base pair is not always preferable to no base pair, which has always been true and requires no new treatment. This analysis can lead to a great simplification. If a base pair can form, and one or the other base is degenerate, then the base pair has a two-fold ambiguity at worst. For example, in the lowest free energy scenario, T:M can only be T:A (as M=AC).

When a base pair can be formed, the possibilities are any or all of C:G, G:C, A:T, T:A, G:T or T:G. For example, N:N allows all six; R:Y could be A:T, G:C, or G:T. Simple base pair stacks are closed by two base pairs. Both interior and hairpin loops require the identity, not only of the closing base pair(s), but also of the neighboring mismatched pair(s). Special rules for “tetra-loops” and “tri-loops” can require special handling.

An array, V(i,j,k), where k=1 or k=2, can be used to contain the minimum free energy for any structure on the sub-fragment, i . . . j, where 1≦i≦j≦n and n is the oligonucleotide length. When a base pair is nondegenerate, k can be set to only equal 1. Otherwise, both k=1 and k=2 can be required to store the minimum free energy corresponding to the two possible choices for the closing base pair. There is no need to allow more possibilities, since it is known a priori that the base pair is either “Strong”, “Weak”, or “Wobble”. Out of 256 possible pairs of degenerate bases, 191 can form valid base pairs. Only 26 of these are degenerate: 16 degenerate “strong pairs”; 9 degenerate “weak pairs”; and a single degenerate “wobble pair” (K:K, where K=GT).

Approximations may be employed in assigning energies to interior and hairpin loops when one or both of the mismatched bases adjacent to a closing base pair is degenerate. A most stable mismatch stacking energy may be used that is a guaranteed lower bound. Single base stacking may be treated in the same manner when a “dangling” base at the end of a helix is degenerate.

A software module can perform a pre-processing step to generate lower bounds for all possible oligonucleotides and oligonucleotide pairs in a gene design project. For example, if folding at 40° C. yields a minimum free energy>0, then that particular oligonucleotide or oligonucleotide pair is guaranteed to melt below 40° C., no matter what base substitutions are made. No further thermodynamics calculations need be done again on such oligonucleotides and oligonucleotide pairs.

### “N−1” Problem

A problem in some embodiments of the disclosed method is that a synthetic oligonucleotide, or small piece of DNA, typically contains a mixture of the desired DNA sequence (“full-length oligonucleotide”) contaminated with sequences with internal point deletions. This problem is referred to herein as the “N−1” problem because oligonucleotides with a single point deletion (“N−1 oligonucleotides”) are the most common contaminant in a typical chemical synthesis of oligonucleotides. Furthermore, in some embodiments, the N−1 oligonucleotides are the most problematic because they are more likely to hybridize, and consequently, to provide undesired products, than oligonucleotides with more than one point deletion or mutation. When this mixture of oligonucleotides is used to synthesize medium and large pieces of DNA as disclosed herein, the product pieces of DNA contain a population containing DNA with the desired sequence as well as DNA with errors arising from incorporation of the N−1 oligonucleotides. The N−1 oligonucleotide errors are cumulative and may cause frame-shift mutations, as understood by those skilled in the art.

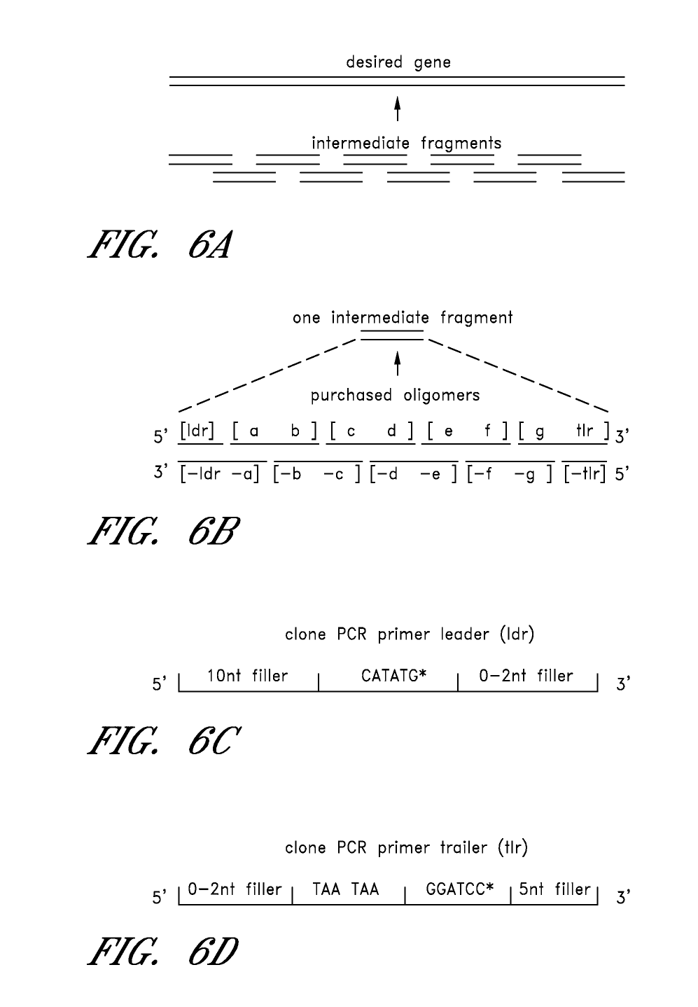

In the chemically synthesized oligonucleotides, the typical coupling efficiency for each nucleotide is from about 98% to about 99.5%, or greater. TABLE I provides the yield of the desired full-length oligonucleotide of length 20 to 250 nt for coupling efficiencies of 99.5%, 99%, and 98%. These results are provided graphically in FIG. 5A-FIG. 5C, respectively. As expected, the probability of synthesizing a correct oligonucleotide decreases with oligonucleotide length and coupling efficiency. Because each of the oligonucleotide pieces used in the construction of the resulting full-duplex DNA contains some N−1 contaminant, the probability of synthesizing the desired DNA decreases with the length of the resulting DNA.

TABLE 1

| Oligonucleotide | Coupling Efficiency | | |
|---|---|---|---|
| Length (nt) | 99.5% | 99% | 98% |
| 20 | 90.916 | 82.617 | 68.123 |
| 25 | 88.665 | 78.568 | 61.578 |
| 30 | 86.471 | 74.717 | 55.662 |
| 35 | 84.311 | 71.055 | 50.334 |
| 40 | 82.243 | 67.573 | 45.480 |
| 45 | 80.208 | 64.261 | 41.11 |
| 50 | 78.222 | 61.112 | 37.16 |
| 55 | 76.286 | 58.117 | 33.59 |
| 60 | 74.398 | 55.268 | 30.363 |
| 65 | 72.557 | 52.56 | 27.445 |
| 70 | 70.761 | 49.984 | 24.808 |
| 75 | 69.009 | 47.534 | 22.425 |
| 80 | 67.301 | 45.204 | 20.27 |
| 85 | 65.635 | 42.989 | 18.323 |
| 90 | 64.011 | 40.882 | 16.562 |
| 95 | 62.427 | 38.878 | 14.971 |
| 100 | 60.881 | 36.973 | 13.533 |
| 110 | 57.905 | 33.438 | 11.057 |
| 115 | 56.472 | 31.799 | 9.995 |
| 120 | 55.074 | 30.240 | 9.034 |
| 130 | 52.381 | 27.239 | 7.382 |
| 140 | 49.821 | 24.734 | 6.031 |
| 150 | 47.385 | 22.369 | 4.928 |
| 160 | 45.068 | 20.23 | 4.027 |
| 170 | 42.865 | 18.296 | 3.29 |
| 180 | 40.769 | 16.546 | 2.688 |
| 190 | 38.776 | 14.964 | 2.196 |
| 200 | 36.88 | 13.533 | 1.795 |
| 210 | 35.08 | 12.24 | 1.47 |
| 220 | 33.36 | 11.07 | 1.19 |
| 230 | 31.73 | 10.01 | 0.98 |
| 240 | 30.18 | 9.05 | 0.8 |
| 250 | 28.7 | 8.19 | 0.65 |

Even in cases in which the desired DNA is synthesized with high probability of correct oligonucleotide order, the desired DNA is invariably mixed with many defective DNA sequences arising from N−1 oligonucleotides. In many applications, this mixture of correct and defective DNA sequences is undesirable. Accordingly, disclosed below is a method for improving the probability of synthesizing the desired DNA and/or selecting the desired DNA from this mixture.

In some embodiments, the N−1 problem is addressed by assembling the chemically synthesized oligonucleotides using direct self-assembly and ligation, as described above and illustrated in FIG. 2. In embodiments using direct self-assembly and ligation, all of the nucleotides in each oligonucleotide are hybridized, thereby reducing the probability that an N−1 oligonucleotide will be incorporated in the preligation DNA construct. In embodiments using overlap extension, a preextension DNA construct incorporating an oligonucleotide with a deletion in a single stranded region is about as likely as a DNA construct incorporating a correct oligonucleotide. The single-base deletion error rate in double-stranded regions is about 0.3%, while the error rate in single-stranded regions is about 0.5%.

In some embodiments, the N−1 problem is addressed by sampling the population of synthetic DNA molecules and sequencing the sampled molecules. In some embodiments, a random sample from the population of different DNA molecules produced in any of the reassembly steps, including the final step, is sequenced and only those molecules with the correct nucleotide sequence are used in the next reassembly step. The optimum sample size is related to the probability of synthesizing the desired DNA molecule. For example, a synthesis of a 200-nt oligonucleotide or intermediate fragment with a 99.5% coupling efficiency provides about 37% of the correct oligonucleotide. Randomly selecting four oligonucleotides or intermediate fragments from the product mixture provides about an 84% chance of selecting at least one correct oligonucleotide. For a 300 nt oligonucleotide or intermediate fragment at 99.5% coupling efficiency, the correct oligonucleotide makes up about 22% of the product. The probability of selecting at least one correct oligonucleotide or intermediate fragment from a sample of four oligonucleotides from this mixture is about 63%. The probabilities of selecting at least one correct oligonucleotide or intermediate fragment using sample sizes of 1, 4, 6, and 8 for syntheses with coupling efficiencies of 99.5% and 99.7% and oligonucleotide lengths of 250 nt, 300 nt, and 300 nt are provided in TABLE II. As shown in TABLE II, only a modest amount of sequencing is necessary to provide a good probability of selecting a correct oligonucleotide or intermediate fragment.

TABLE II

| Oligonucleotide Length | Coupling | Sample Size | | | |
|---|---|---|---|---|---|
| (nt) | Efficiency | 1 | 4 | 6 | 8 |
| 200 | 99.5% | 36.7 | 83.9 | 93.6 | 97.4 |
| | 99.7% | 53.7 | 95.4 | 99.0 | 99.7 |
| 250 | 99.5% | 28.6 | 74.0 | 86.7 | 93.2 |
| | 99.7% | 46.0 | 91.5 | 97.5 | 99.3 |
| 300 | 99.5% | 22.2 | 63.4 | 77.9 | 86.6 |
| | 99.7% | 39.4 | 86.5 | 95.0 | 98.2 |

In some embodiments, sampling is performed by cloning the DNA-to-be-sequenced into a suitable vector. Typically, each transformed colony corresponds to one molecule of the synthetic DNA. In some embodiments, a sample of transformed colonies are selected, the DNA sequenced, and DNA with the correct sequence is used in the next hierarchical stage of assembly. The cloning is any type of cloning known in the art. In one embodiment, the cloning is topoisomerase I (TOPO®, Invitrogen) cloning. The sampling is performed at any of the reassembly stages.

In some embodiments, the N−1 problem is addressed by analyzing the polypeptide(s) expressed from a sample from the population of synthetic DNA sequences. The DNA is expressed using any means known in the art, for example, inserting the gene in an expression vector or using a cell-free expression system. In some embodiments, an organism is transformed by electroporation or using a gene gun. In some embodiments, the DNA sequence is cloned in an expression vector and expressed. As discussed above, each clone typically corresponds to one DNA molecule from the population. In some embodiments, the DNA is the full-length synthetic gene. In other embodiments, the DNA is an intermediate fragment. In the case of an intermediate fragment, those skilled in the art will realize that, in some embodiments, the intermediate fragment is designed with (1) a leader that provides a start codon in the correct reading frame, that is, provides an ATG in the DNA and a 0-2 nt filler that adjusts the reading frame in order to express the desired polypeptide, and (2) a trailer that provides one or more stop codons (TAA, TAG, or TGA) in the DNA and a 0-2 nt filler that adjusts the reading frame in order to terminate the desired polypeptide. Typically, the reading frame is the same as for the full-length synthetic gene, although other reading frames are used in some embodiments. Typically, from zero to two bases are inserted into the leader and trailer for adjusting the reading frame. Those skilled in the art will recognize that more than two bases could be used to adjust the reading frame. For example, in some embodiments, the leader and/or trailer encodes additional amino acids, restriction sites, or control sequences. Those skilled in the art will further realize that, in some embodiments, different leaders and/or trailers are used in conjunction with the same piece of DNA in different steps of the method. For example, in some embodiments, the leader and/or trailer used in the expression of a polypeptide from a piece of DNA is different from the leader and/or trailer used in the assembly of that piece of DNA. Some embodiments, provide one or more stop codons downstream (3′) of the gene in order to stop the translation of DNA fragments constructed from one or more N−1 oligonucleotides. In some embodiments, the stop codons are engineered into the expression vector. Some embodiments include at least three stop codons downstream (3′) of the gene, at least one of each in each of the three possible reading frames. Some embodiments use groups of stop codons instead of single stop codons in each reading frame.

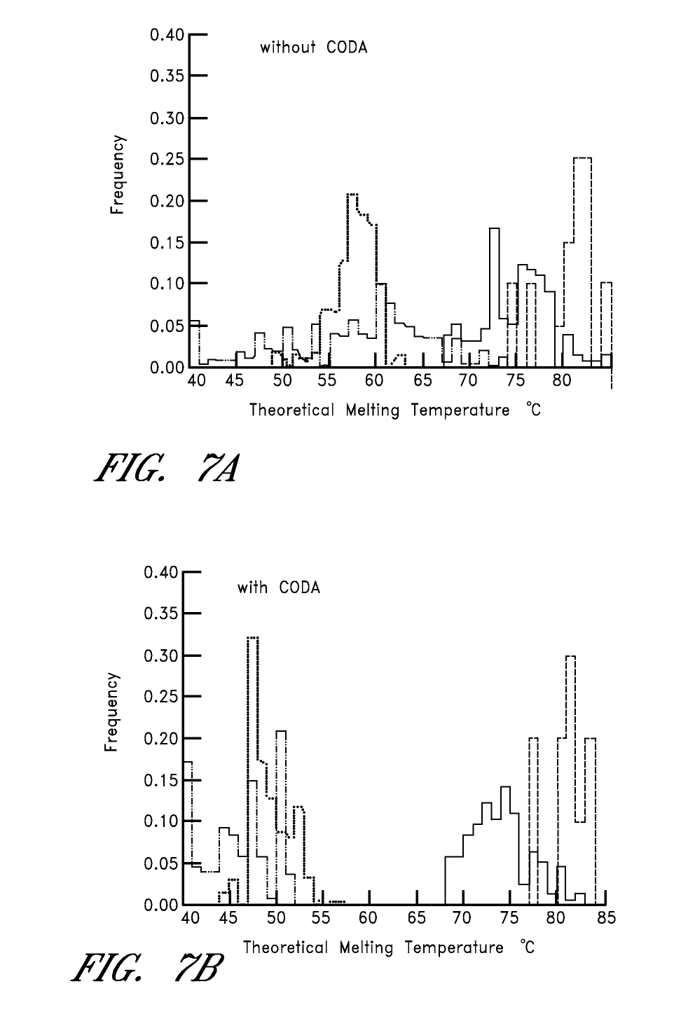

FIG. 6A—FIG. 6D illustrate an embodiment of the disclosed method in which a polypeptide is expressed from an intermediate fragment in the construction of a synthetic gene. FIG. 6A illustrates schematically the division and construction of a gene into a plurality of intermediate fragments.

FIG. 6B illustrates the division and construction of one of the intermediate fragments. The letters a-g each represents a portion of the sequence of the intermediate fragment. The brackets group these portions into oligonucleotides that are purchased or synthesized. “ldr” and “tlr” represent a leader and trailer, respectively. The corresponding portions of the sequence on the complementary strand are prefixed with a hyphen (-), i.e., “-ldr,” “-a,” . . . “-g,” and “-tlr.” Again, brackets are used to indicate the oligonucleotides.

FIG. 6C is a schematic of leader (ldr) portion illustrated in FIG. 6B. From the 5′-end, the leader comprises a 10-nt filler, a CATATG restriction site, and a 0-2-nt filler at the 3′-end. In the illustrated embodiment, the length of the 5′-filler is determined by the requirements of the restriction enzyme. The restriction site is used in cloning the intermediate fragment, and includes an ATG start codon. The 0-2-nt filler adjusts the reading frame of the intermediate fragment relative to the start codon. In some embodiments, the restriction site does not include a start codon. In some embodiments, a start codon is incorporated in the 3′-filler.

FIG. 6D is a schematic of the trailer (tlr) portion illustrated in FIG. 6B. From the 5′-end, the trailer comprises a 0-2-nt filler, a TAATAA stop sequence, a GGATCC restriction site, and a 5-nt filler. The 0-2-nt filler adjusts the reading frame of the stop codon relative to intermediate fragment. TAATAA is a pair of stop codons. Any suitable stop codon is useful. Some embodiments use one stop codon. GGATCC is a restriction site used for cloning the intermediate fragment. The length of the 3′-filler is determined by the requirements of the restriction enzyme. Those skilled in the art will understand that, in other embodiments, the leader and/or trailer use a different combination of fillers, restriction sites, start codon, and/or stop codons. In some embodiments, the intermediate fragment comprises a start and/or stop codon and the leader and/or trailer does not include the codon. For example, in the case of a synthetic gene, the gene typically includes both a start and stop codon. Similarly, those skilled in the art will understand that in some embodiments, the leader and/or trailer does not comprise a restriction site. Some embodiments of the leader and/or trailer do not use a 5′- and/or a 3′-filler.

A polypeptide expressed from a clone with an N−1 defect will be defective. The expressed peptide is analyzed using any means known in the art, for example, gel electrophoresis, capillary electrophoresis, two-dimensional electrophoresis, isoelectric focusing, spectroscopy, mass spectroscopy, NMR spectroscopy, chemically, ligand binding, enzymatic cleavage, or a functional or immunological assay. A clone that expresses the correct peptide is free from N−1 defects.

In some embodiments, the expressed polypeptide is analyzed using gel electrophoresis, which separates polypeptides by molecular weight. Of the 64 possible DNA codons, 3 are stop codons. Consequently, the frame-shift caused by a point deletion is likely to generate a new stop codon, resulting in a prematurely truncated polypeptide, the molecular weight of which is determined using gel electrophoresis. A clone that provides a full-length polypeptide is likely to have the desired sequence, while one that provides a truncated polypeptide is likely to have at least one point deletion. In some embodiments, a clone with an N−1 defect or defects produces a polypeptide that is too long, because the N−1 defect results in a frame-shift that causes the terminating stop codon(s) to be ignored (read through). In some embodiments, such a polypeptide that is too long will be terminated by a stop codon engineered into the expression vector downstream (3′) of the gene. As discussed above, some embodiments comprise three groups of stop codons, one group in each possible reading frame. In these embodiments, the molecular weight of the expressed polypeptide is higher than expected.

In some embodiments, analysis of the expressed polypeptide is used to narrow the sample of clones that are then sequenced. In these embodiments, the analysis of the expressed polypeptide is used to identify and to eliminate clearly defective (e.g., truncated or too long) DNA clones. The remaining clones are then sequenced. In these embodiments, the expression and analysis is a semi- or nonrandom selection method, in contrast to the random selection method described above. In some embodiments, the expressed polypeptide is analyzed by gel electrophoresis. In some cases, gel electrophoresis does not distinguish a defective polypeptide from the correct polypeptide. For example, in some cases a DNA sequence with an N−1 defect generates a defective polypeptide that, to within the resolution of the electrophoresis conditions, has the same molecular weight as the correct polypeptide. This scenario can arise where the defective DNA sequence fortuitously expresses a defective polypeptide similar in molecular weight to the correct polypeptide, for example, where the point defect is near the end of the clone. In another scenario, the clone has 3N point deletions that do not generate a new stop codon. As discussed above, the defective polypeptide is most likely shorter than the correct polypeptide. A defective polypeptide closer in molecular weight to the correct polypeptide than the resolution of the electrophoresis experiment is not distinguished. Given the resolving ability of gel electrophoresis, selecting a correct clone using the method is highly probable. The probability is further improved using an analytical technique with higher resolution, for example, capillary electrophoresis or mass spectroscopy. In some cases, all of the clones selected for sequencing in the gene expression screen have the correct sequence, indicating the reliability of this selection method. Furthermore, expressing a gene and determining the molecular weight of the expressed polypeptide is typically faster and/or less expensive than the equivalent amount of DNA sequencing. In some embodiments intermediate fragments are selected by estimating the molecular weight of the expressed polypeptide only, and DNA sequencing is reserved only for the final gene construct, and even then only after its molecular weight of a polypeptide expressed from the final gene has been estimated to be correct.

In some embodiments, all of the expressed polypeptides that are analyzed are defective, for example, truncated. In these embodiments, an analysis of the defective polypeptides indicates the location of the defect in the DNA sequence. The gene is then resynthesized using this information. In embodiments using multiple hierarchical synthesis steps, only some of the pieces of DNA are resynthesized, for example, an intermediate fragment containing the defect. In some embodiments, the offending fragment is divided in a different way and/or reoptimized, as discussed above. In some embodiments a different clone is chosen to replace the offending fragment.

In some embodiments, the DNA sequence is transformed into a selected organism such that a clone containing a correct and/or likely-to-be correct DNA sequence will exhibit a phenotype different from the phenotype exhibited by a clone containing an incorrect DNA sequence. The organism is a prokaryote or a eukaryote. Examples of suitable prokaryotes include bacteria, for example, E. coli. Examples of suitable eukaryotes include yeast, fungi, and mammalian cells. The differences in phenotype arise from mechanisms well known in the art, for example, differential induction and/or repression of gene expression by correct and incorrect DNA sequences, expression of different proteins or polypeptides by the correct and incorrect DNA sequences, and the like. In some embodiments, the difference in phenotype is detectable without specialized equipment, for example, by inspection by the naked eye. For example, in some embodiments, the difference in phenotype is the color of the organism. In other embodiments, the difference in phenotype is viability of the organism under particular conditions. Examples of particular conditions include pH, temperature, light, and the like. In some embodiments, conditions include the presence or absence of a particular compound or compounds, including, nutrients, for example, amino acids, carbohydrates, vitamins, cofactors and the like; and/or antibiotics or other toxic compounds. Those skilled in the art will understand that other particular conditions are compatible with the disclosed method. Those skilled in the art will understand that the embodiments described below are exemplary only, and that the method may be varied to use, for example, other organisms, phenotypes, conditions, genes, and vectors.

Some embodiments of the method use a vector referred to herein as a “frameshifted vector” to distinguish between correct and incorrect DNA sequences. The frameshifted vector is any type of vector known in the art useful for introducing a gene into an organism, for example, a plasmid, a cosmid, a phagemid, a bacteriophage, a virus, and/or a bacterium. The frameshifted vector contains a gene, that when expressed, changes the phenotype of a selected organism into which the vector is transformed, for example, color or viability. In the frameshifted vector, a frameshift is introduced into at least a portion of the open reading frame (ORF) of the gene such that the gene does not express a functional product. In some embodiments, the frameshift is introduced upstream of a functional portion of the gene. A functional portion of the gene is a portion that expresses a functional polypeptide or protein, the expression of which changes the phenotype of the organism. When an organism is transformed using the frameshifted vector, no functional product is expressed, and consequently, no change in phenotype is observed. The term “frameshift” as used herein is used in its usual sense, as well as to mean the insertion and/or deletion of one or more bases, which results in a change in reading frame. The three possible reading frames for the gene are referred to herein as the correct reading frame; the +1 or n+1 reading frame; and the −1 or n−1 reading frame. In a frameshifted vector, at least a portion of the ORF is in the −1 or +1 reading frame. Those skilled in the art will understand that for any particular vector/organism system, two frameshifted vectors, one with a −1 reading frame and one with a +1 reading frame are sufficient to perform the disclosed method with any synthetic DNA sequence designed to shift the downstream reading frame. In some embodiments, the frameshifted vector also includes at least one DNA insertion site upstream of the region of the gene that encodes the functional portion of the protein or peptide. The DNA insertion site is of any type known in the art useful for inserting a piece of DNA into the frameshifted vector. In some embodiments, the DNA insertion site is one or more restriction sites. Those skilled in the art will understand that any compatible restriction site known in the art is suitable. Examples of suitable restriction sites include, EcoR I, BamH I, Hind III, Pci I, Age I, Spe I, Nde I, Nco I, Sac I, Sac II, Pvu I, Xho I, Pst I, and Sph I. Some embodiments of the frameshifted vector comprise a plurality of DNA insertion sites.

The synthetic DNA sequence is designed to correct the frameshift when inserted into the DNA insertion site. In other words, the DNA sequence is designed with a length that corrects the −1 or +1 shift designed into the reading frame of the functional portion of the gene. A piece of DNA with the desired sequence is also referred to herein as having a “correct” DNA sequence. Consequently, the functional portion of the gene is in the correct reading frame when a correct DNA sequence is inserted therein. On transforming the selected organism with the resulting vector, the organism expresses functional polypeptide or protein, which changes the phenotype of the organism.

In contrast, when a DNA sequence with an N−1 defect is inserted into the DNA insertion site, the frameshift is not corrected. For example, for a vector with a −1 frameshift, inserting an N−1 DNA sequence produces a +1 frameshift. Inserting a DNA sequence with two N−1 defects also does not correct the frameshift. For a vector with a −1 frameshift, inserting a DNA sequence with two N−1 defects produces a −1 frameshift. Inserting a DNA sequence with three N−1 defects corrects the frameshift. In general, a DNA sequence with 3n N−1 defects will correct the frameshift, while those with 3n+1 or 3n+2 N−1 defects will not. Given the low error rates for incorporation of N−1 oligonucleotides in the synthesis of the next-larger piece of DNA provided above, especially for ligation-based methods, the probability that a DNA sequence will have three or more N−1 defects is low, although not negligible. Consequently, most of the DNA sequences with the correct reading frame in the frameshifted vector have the correct sequence. In some embodiments for selecting intermediate fragments, about 80% to about 95% of clones exhibiting the changed phenotype have the correct DNA sequence. The remainder have three or more N−1 defects, which is consistent with the error rates provided above.

Similarly, when an organism is transformed with a frameshifted vector into which no DNA is inserted, the frameshift engineered into the vector causes no functional product to be expressed, and consequently, no change in phenotype. Of the four most likely DNA inserts into the frameshifted vector—no DNA, DNA with one N−1 defect, DNA with two N−1 defects, and correct DNA—only the vector with the correct DNA inserted therein changes the phenotype of an organism transformed therewith. An organism exhibiting the changed phenotype is selected, the correct DNA sequence isolated, and the DNA sequence used as described herein. The correct DNA sequence is isolated by any method known in the art, for example, by excision or by PCR using suitable primers.

A frameshifted vector is synthesized by any method known in the art. Some embodiments use known combinations of a particular vector and organism such that the organism changes phenotype when transformed with the vector. Typically, the vector has an open reading frame containing a functional portion of a gene, the expression of which changes the phenotype of the organism. One or more bases are inserted and/or deleted in the ORF upstream of the functional portion of the gene, thereby causing a −1 or +1 frameshift in the functional portion of the gene. The bases are inserted and/or deleted by methods known in the art, for example, cutting with restriction enzymes, digestion of double- or single-stranded portions, site-directed mutagenesis, ligation, chemical synthesis, and the like. In some embodiments, a DNA insertion site is also engineered upstream of the functional portion of the gene. In other embodiments, the ORF contains a preexisting DNA insertion site upstream of the functional portion of the gene.

Some embodiments distinguish a correct DNA sequence from an incorrect DNA sequence by the color of the transformed organism. An embodiment of the method uses a vector with a gene encoding the α-complementing fragment (α-fragment) of E. coli lacZ β-galactosidase. The DNA sequence is inserted at the DNA insertion site located upstream of the functional portion of the gene for the α-complementing fragment. The vector is engineered so that functionality of the α-fragment gene depends on the reading frame of the functional portion of the gene after the synthetic DNA sequence is inserted into the DNA insertion site. The vector is transformed into an E. coli strain containing a 5′-truncation of the lacZ gene. Protein expressed by a functional α-fragment gene transcomplements the defective lacZ expressed by the cell, thereby producing functional β-galactosidase. When the cells are grown on indicator media containing isopropylthio-β-D-galactoside (IPTG) and 5-bromo-4-chloro-3-indolyl-β-D-galactoside (X-Gal), colonies developing from cells with a functional α-complementing fragment gene are blue, while those with a defective α-complementing fragment gene are white.

In one embodiment, the frame-shifted vector is a modified pGEM®-3Z vector (Promega Corp., Madison, Wis.). The pGEM®-3Z vector is a pBR322-based plasmid that contains a multiple cloning site (MCS) in the ORF of the α-fragment gene, as well an ampicillin resistance gene. The vector is engineered with a frameshift mutation in the α-fragment gene, which renders the gene non-functional. The DNA sequence is designed to correct the frameshift when inserted at the MCS, thereby producing a functional α-fragment gene. Colonies of cells transformed with the frameshifted vector are white. White colonies are also observed for cells transformed with a frameshifted vector into which a DNA sequence with one or two N−1 defects is inserted. Blue colonies are observed only for cells transformed with a frameshifted vector into which a DNA sequence with no defects is inserted. A feature of this system is that the α-fragment is known to retain its activity with up to 650 amino acid extensions at the N-terminus.

In some embodiments, the difference in phenotype is temperature resistance. Some embodiments use E. coli AB4141 (F, metC56, lct-1, thi-1, valS7, ara-14, lacY1, galK2, xyl-7, rpsL69, tfr-5, supE44), which contains a conditionally lethal, temperature sensitive valS (valyl-tRNA synthetase). This strain grows at a permissive temperature of about 37° C., but not at a restrictive temperature of about 42° C. After transformation with a plasmid expressing wild-type valS, the strain grows at the restrictive temperature. One embodiment uses a frameshifted vector derived from the plasmid pDH-1Δ11, which includes a wild-type valS gene. In this system, any colony growing at the restrictive temperature has the correct DNA sequence. Cells without the correct DNA sequence do not grow at all at the restrictive temperature.

Some embodiments provide a kit comprising one or more frameshifted vectors and instructions for using the frameshifted vector(s) to isolate a DNA sequence as described herein. Some embodiments of the kit also include other components, for example, a preselected organism, a growth medium, a restriction enzyme, and the like.

As used herein, the term DNA includes both single-stranded and doubled-stranded DNA. The term piece refers to either a real or hypothetical piece of DNA depending on context. A very large piece of DNA is longer than about 1,500 bases, a large piece of DNA is about 1,500 bases or fewer, a medium-sized piece of DNA is about 300 to 350 bases or fewer, and a short piece of DNA is typically less than 300 bases and can be about 50 to 60 bases or fewer. It will be appreciated that these numbers are approximate, however, and may vary with different processes or process variations. Although descriptions of preferred embodiments of the disclosed method describe each recursive or hierarchical step as involving pieces of DNA of the same size range—for example, in which all of the pieces of DNA are very large, large, medium-sized, or short—one skilled in the art will appreciate a hierarchical step may involve DNA from more than one size range. A particular step may involve both short and medium-sized pieces of DNA, or even short, medium-sized, and large pieces of DNA.

A small or short piece of DNA is a DNA segment that can be synthesized, purchased, or is otherwise readily obtained. The term segment is also used herein to mean small piece. Those skilled in the art will understand that the term synthon is synonymous with the terms small piece and segment as used herein, although the term synthon is not used herein. The DNA segments used can be synthetic; however, the disclosed method also comprehends using DNA segments derived from other sources known in the art, for example, from natural sources including viruses, bacteria, fungi, plants, or animals; from transformed cells; from tissue cultures; by cloning; or by PCR amplification of a naturally occurring or engineered sequence. As used herein, a correct piece of DNA is a piece of DNA with the correct or desired nucleotide sequence. An incorrect piece is one with an incorrect or undesired nucleotide sequence.

A synthetic oligonucleotide can be in a mixture containing the desired oligonucleotide mixed with incorrect oligonucleotides, that is, oligonucleotides that do not have the desired sequence. As would be apparent to one skilled in the art, synthesizing a gene from such a mixture will likely produce the correct or desired gene in admixture with incorrect genes. One method for synthesizing only the correct gene is to assemble the gene from multiple DNA sequences that, combined, are likely to have the correct sequences. Consequently, in some embodiments, during the assembly process, pieces of DNA are selected that are likely to have the correct sequences for use in subsequent assembly steps. In some embodiments, the criterion for the selection is a property of an assembled piece of DNA or a polypeptide encoded by and expressed therefrom. In some embodiments, the criterion for the selection is a property determined from the full-length piece of DNA or polypeptide expressed therefrom. In some embodiments, the criterion for the selection is a property determined for the complementary strand of DNA or polypeptide expressed therefrom. In some embodiments, the criterion for the selection is a property determined for a piece of RNA transcribed from the piece of DNA. In some embodiments, the criterion for the selection is the phenotype of an organism into which the DNA is inserted.

The term PCR as used herein in the context of assembling or reassembling DNA is a PCR or overlap extension reaction, preferably using a proof-reading DNA polymerase (proof-reading PCR). The term direct self-assembly as used herein in the context of assembling or reassembling DNA is a copy-free method of producing a DNA construct or a DNA construct produced by the method, comprising assembling a large piece of DNA from short synthetic segments in a single step. Copy-free means that the method lacks a copy step, such as is found in overlap extension or PCR, thus eliminating the copying errors. In a preferred embodiment, adjacent segments on the same strand abut, i.e., form a nick in the strand. Preferably, the nicks in the self-assembly are repaired by in vitro ligation. In another preferred embodiment, the nicks are repaired in vivo by cellular machinery after cloning.

The terms set and group as used herein is refers to a collection of two or more.

### EXAMPLES

### Example 1

### Melting Temperature Probability Distributions of Correct and Incorrect Hybridizations

This example illustrates the synthesis of an E. coli threonine deaminase gene by a two-step hierarchical decomposition and reassembly by overlap extension. E. coli threonine deaminase is a protein with 514 amino acid residues (1,542 coding bases).

### Design

The sequence design method permuted synonymous (silent) codon assignments to each amino acid in the desired protein sequence. Each synonymous codon change results in a different artificial gene sequence that encodes the same protein. Because E. coli was the desired expression vector, the initial codon assignment was to pair each amino acid with its most frequent codon according to E. coli genomic codon usage statistics. Subsequently, the codon assignments were perturbed as described below. The final codon assignment implied a final DNA sequence to be achieved biochemically.

In this two-step hierarchical decomposition, the gene was divided first into five overlapping medium-sized pieces (in the present example, not longer than 340 bases, overlap not shorter than 33 bases), then each medium-sized piece was divided into several overlapping short segments (in the present example not longer than 50 bases, overlap not shorter than 18 bases). All overlaps were lengthened if necessary to include a terminal C or G for priming efficiency.

Theoretical melting temperatures were calculated for correct and incorrect hybridizations of both the un-optimized and the melting-temperature optimized oligonucleotides and intermediate fragments. Theoretical melting temperatures were calculated with Mfold (13) for [Na+]=0.01M and [Mg++]=0.0015M with folding temperature=50° C. Every amino acid was assigned its most frequent codon in E. coli highly expressed genes.

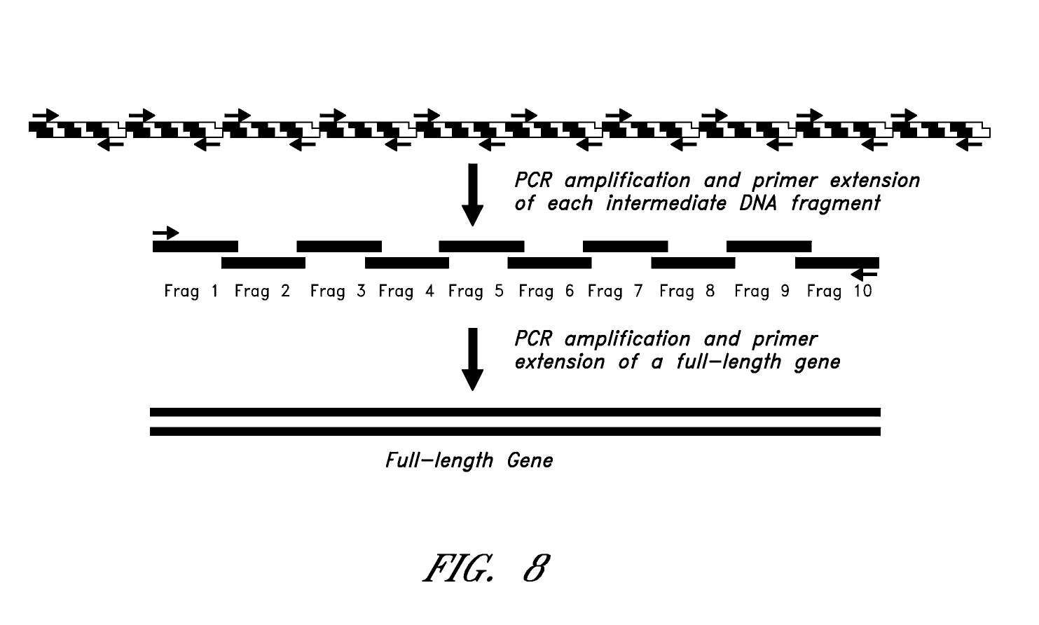

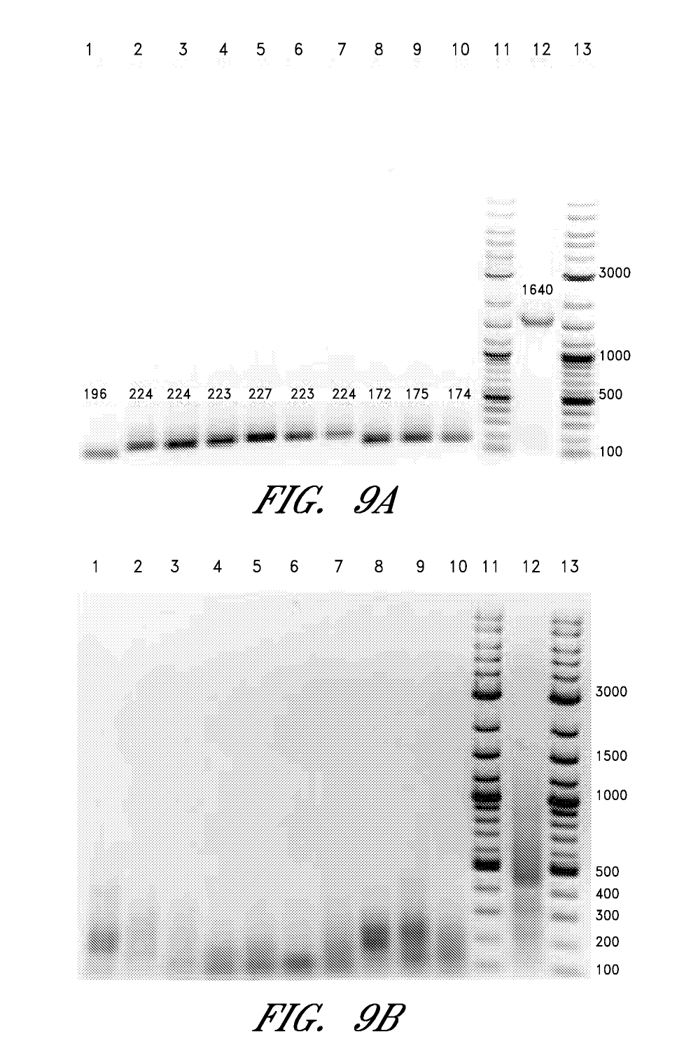

FIG. 7A shows the probability distribution of theoretical melting temperatures of un-optimized oligonucleotides and intermediate fragments. Solid and dashed lines represent correct hybridizations of oligonucleotides and intermediate fragments, respectively. Dot-dashed and dotted lines represent incorrect hybridizations of oligonucleotides and intermediate fragments, respectively. The melting temperatures of the incorrect hybridizations overlap with those of the correct hybridizations for the un-optimized oligonucleotides and fragments.

FIG. 7B shows the probability distribution of theoretical melting temperatures of the melting-temperature optimized oligonucleotides and intermediate fragments. Lines are as defined for FIG. 7A. In this case, the melting temperatures of the incorrect hybridizations are separated by a melting temperature gap of 18° C. from the correct hybridizations.

### Example 2

### Accuracy of Gene Assembly

A first set of overlapping abutting oligonucleotides of the integrase (IN) gene was generated that was optimized for E. coli codon usage and self-assembly and was melting-temperature optimized by requiring the minimum melting temperature for every correct overlap hybridization event is 10° C. to 20° C. higher than the maximum melting temperature for any mismatch. For comparison, a second set of overlapping abutting oligonucleotides was generated that was optimized for E. coli codon usage but was not melting-temperature optimized. For both sets, the full-length IN gene was assembled from other identical oligonucleotides and fragments by the assembly process diagrammed in FIG. 8.

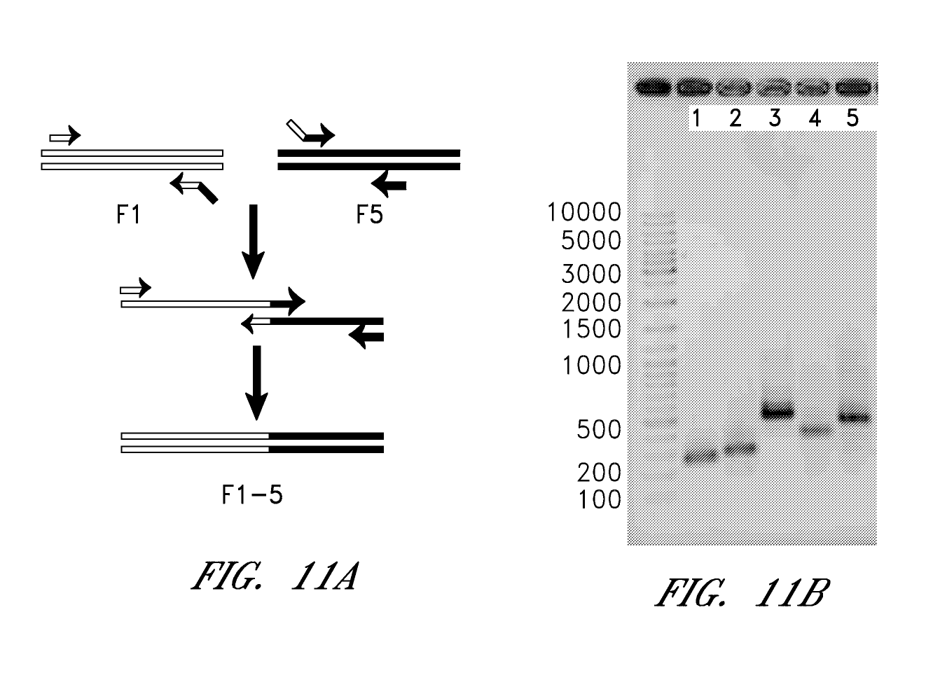

First, each intermediate fragment was assembled with six to eight oligonucleotides approximately 50 nts in length (fragment 0: 196 bp, 8 oligonucleotides; fragment 1: 224 bp, 8 oligonucleotides; fragment 2: 224 bp, 8 oligonucleotides; fragment 3: 223 bp, 8 oligonucleotides; fragment 4: 227 bp, 8 oligonucleotides; fragment 5: 223 bp, 8 oligonucleotides; fragment 6: 224 bp, 8 oligonucleotides; fragment 7: 172 bp, 6 oligonucleotides; fragment 8: 175 bp, 6 oligonucleotides; fragment 9: 174 bp, 8 oligonucleotides). These constituent oligonucleotides of each intermediate DNA fragment were mixed and added to a primer extension reaction at a final concentration of 0.1 μM. In addition, an excess of the leader and the trailer oligonucleotides was added to the assembly reaction at final concentration of 1 μM. Ten, the oligonucleotides were extended to the full length of each intermediate fragment with DNA polymerase in a primer extension and PCR amplification reaction (FIG. 7 and FIGS. 9A and 9B, lanes 1-10). The intermediate DNA fragments were mixed and added to a primer extension reaction at a final concentration of 0.1 μM. In addition, an excess of the gene leader and trailer oligonucleotides was added to the assembly reaction at final concentration of 1 μM. Finally, the overlapping intermediate DNA fragments were extended to the full-length gene in a primer extension and PCR amplification reaction (FIGS. 9A and 9B, lane 12)

FIG. 9A shows the electrophoretogram of assembly of intermediate DNA fragments assembled from oligonucleotides optimized for codon usage and melting-temperature optimized. Lanes 1-10 show IN intermediate DNA fragments 0 through 9, assembled from melting-temperature optimized oligonucleotides. Lanes 11 and 13 are molecular weight markers. Lane 12 shows the complete, full-length IN gene (1,640 bp) from the melting-temperature optimized intermediate IN DNA fragments. A sharp band is evident, indicating that the IN gene was successfully assembled. (The accuracy of the assembly of the full-length IN gene was verified by DNA sequencing of both strands of the IN gene in a pET-3a vector, and by in vivo expression of a protein product of the correct molecular weight in an E. coli BL21-DE3 strain, not shown.)

FIG. 9B shows the electrophoretogram of assembly of intermediate DNA fragments assembled from oligonucleotides optimized for codon usage but not melting-temperature optimized. Lanes are as described above. In this case, no DNA band is visible in Lane 12 at the position expected.

### Example 3

### Pooled Oligonucleotides

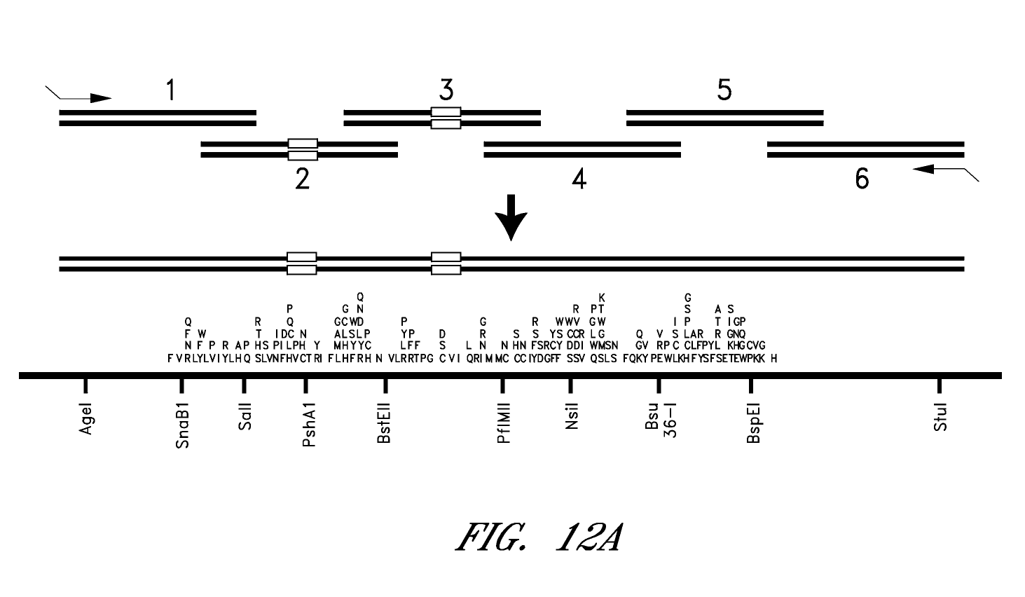

The 1,640 by Ty3 IN gene was divided into fragments that were melting-temperature optimized by requiring the minimum melting temperature for every correct overlap hybridization event is 10° C. to 20° C. higher than the maximum melting temperature for any mismatch. The 42 oligonucleotides for the seven non-overlapping, odd-numbered, intermediate DNA gene fragments were assembled in one tube, and the 48 oligonucleotides for the eight even-numbered intermediate DNA gene fragments were assembled in a second tube. FIG. 10A shows the electrophoretogram of the mixtures. To demonstrate that the single bands in FIG. 10A were an exact mixture of all desired even- or odd-numbered intermediate DNA gene fragments, terminal oligonucleotides were used for PCR amplification of each fragment individually. FIG. 5B shows the electrophoretogram of the assembled fragments. The sharp bands indicate clean independent assembly of multiple intermediate DNA gene fragments from a single pool of oligonucleotides. The overlapping intermediate DNA fragments were extended to the full-length gene in a primer extension and PCR amplification reaction. The sharp band in FIG. 10C indicates clean correct self-assembly of the 1,640 by full-length yeast Ty3 IN gene.

### Example 4

### Directed Shuffling (Rearrangement) of DNA Sequences

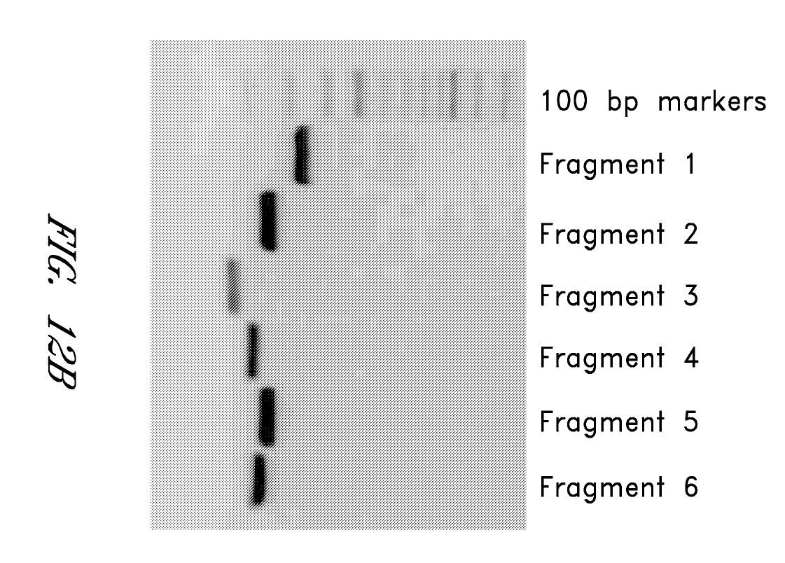

Two primers were designed that contained sequences complementary to one another as well as sequences complementary to intermediate DNA melting-temperature optimized fragments F1 and F5 for the 1,640 by Ty3 In gene, such that fragments F1 and F5 could be combined into a single fragment after a primer extension reaction (FIG. 11A). These two primers, the leader primer of fragment F1, and the trailer primer of fragment F5 were extended in a reaction mixture in the presence of all of the odd-numbered intermediate DNA fragments for the 1,640 by Ty3 In gene. The sharp band in Lane 1 of the electrophoretogram of FIG. 118 indicates that this reaction produced a single correctly rearranged 259 by chimeric DNA fragment containing the desired subsequences from fragment F1 and fragment F5.

Another reaction employed the same process to produce a 282 by chimeric fragment from intermediate DNA melting-temperature optimized fragments F0 and F6 for the 1,640 by Ty3 In gene (FIG. 11B, Lane 2). Since the trailer primer sequence of fragment F0-F6 ((FIG. 11B, Lane 2) was designed to have sequences complementary to the leader primer sequence of fragment F1-F5 ((FIG. 11B, Lane 1), it was possible to extend these rearranged fragments to produce a longer 498 by F0-F6-F1-F5 DNA fragment (FIG. 11B, Lane 3). In the same manner, a 383 by rearranged DNA fragment was assembled from three fragments, F1 F5 and F9 (FIG. 11B, Lane 4), as well as a 462 by rearranged DNA fragment from DNA fragments F0, F6 and F8 (FIG. 11B, Lane 5).

### Example 5

### Point Mutations

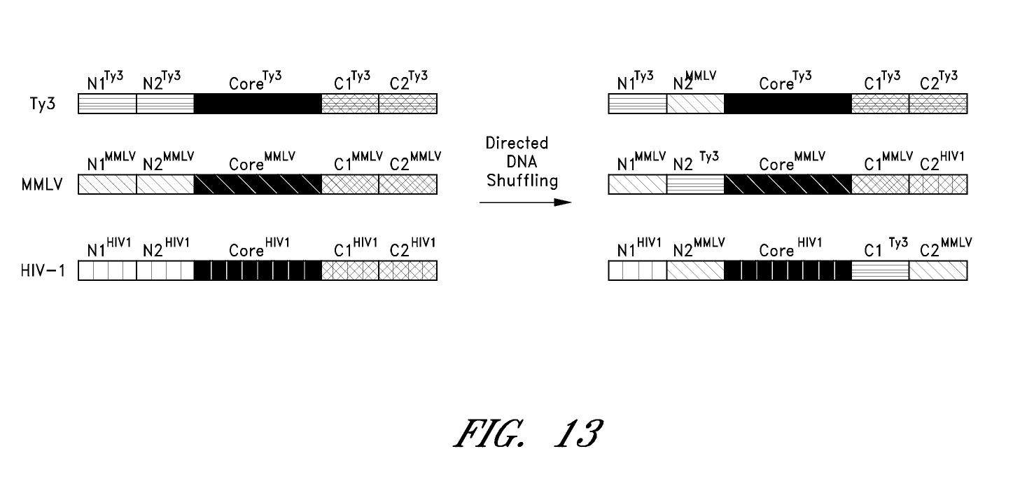

The tumor suppressor p53 gene was divided into DNA fragments and then into oligonucleotides. The oligonucleotides were optimized as described above, such that internal sequences of the p. 5.3 gene were uniquely thermodynamically addressable. Oligonucleotides were allowed to self-assemble to form DNA constructs, which were extended to form DNA fragments. Clean self-assembly of the DNA fragments were verified by the sharp bands of the electrophoretogram of FIG. 12B.

Primers are synthesized that are complementary to specific sequences of the DNA fragments. The primers attach to the DNA fragments and cause specific desired point mutations (FIG. 12A, top row). The DNA fragments are then assembled to form a mutant p53 gene (FIG. 12A, middle row).

### Example 6

### Gene Synthesis

DNA molecules are globally optimized and oligonucleotides complementary to internal sequences are used to produce directed sets of rearranged DNA sequences. This advance was possible because each oligonucleotide is globally optimized to hybridize only to its adjacent overlapping oligonucleotides. DNA molecules are the 1,640 by integrase (IN) gene and/or the GAG3 encoding region of the yeast retrotransposon Ty3. Ty3 is a retroviruslike element in Saccharomyces cerevisiae which replicates and integrates through a cycle similar to that of mammalian retroviruses. Ty3 is distinctive among all retroviruses and retroviruslike elements in that it inserts, with position specificity, at RNA polymerase III transcription initiation sites. In vitro experiments recently have indicated that this specificity is conferred by interactions between integrase and RNA polymerase III transcription factor TFIIIB. These protein interactions direct Ty3 integration to RNA polymerase III transcribed promoter regions. The Ty3 integrase protein sequence shares sites conserved among mammalian retrovirus integrase sequences, including the amino terminal zinc binding domain and other active sites. However, the Ty3 integrase amino- and carboxyl-terminal domains have extensions of approximately 100 aa and 200 aa respectively. Methods and compositions described herein are used to further map the interactions responsible for Ty3 integrase targeting.

A method for targeting retrovirus vector insertions with Ty3 specificity to RNA polymerase III transcription units (e.g., tRNA and 5S genes) can have several potential advantages for gene therapy applications in humans. For example, targeting should be relatively efficient because RNA polymerase III transcription units are redundant. In addition, insertions are non-disruptive because Ty3 inserts at the transcription initiation site and many RNA polymerase III promoter sequences are internal. Thus, the development of a retrovirus with Ty3 targeting specificity would constitute a significant gene therapy advance because insertions would likely have more predictable expression.

In previous work, researchers performed domain swaps between Ty3 and the Moloney Murine Leukemia Virus (MMLV) with limited success. However, more recent work has been successful in swapping integrases between different retroviruses such as MMLV and HIV-1. Therefore, methods and compositions described herein are used to design directed mutant gene sets for efficient expression in human cells of various chimeric Ty3, MMLV, and HIV-1 integrase proteins.

Integrases can be divided into five domains: NTD1, NTD2, core, CTD1, and CTD2. 75 possible combinations of the five integrase domains are created. These constructs are expressed in a human lentiretrovirus packaging cell line and evaluated for (1) integrase expression; (2) ability of viruses to produce cDNA; and (3) ability to integrate.

It is also of interest to determine the Ty3 GAG3 protein that are important for its assembly into an icosahedral viruslike particle and its interactions with host proteins. To aid in these studies, technology described herein is used to construct of a set of mutations that replace each charged amino acid residue with alanine. Again, this directed mutant gene set is expressed from a galactose-inducible promoter on a yeast plasmid vector and assay particle assembly by density gradient centrifugation and atomic force microscopy.

Additionally, identification of GAG3 protein regions important for assembly, such as those required for genomic RNA interaction or for hexamer and pentamer formation, are screened with a directed mutant gene set of small insertions and deletions. Appropriate positions for the mutations are provided.

### Point Mutations: Construction of an Alanine Scanning Mutation Gene Set for the Ty3 GAG3 Protein

The codons for each charged amino acid residue of the Ty3 GAG3 protein are replaced with a codon for alanine. Two oligonucleotides of 45 nt to 50 nt are designed to encode an alanine codon directed to each charged amino acid codon of the globally optimized gene. These oligonucleotides are directed the codon replacement to the correct site in the gene. Next, two intermediate DNA sequences are created by primer extension and PCR amplification. The two resulting DNA gene fragments overlap by 45 to 50 nucleotides. Finally, they are primer extended and PCR amplified into the full-length gene, as illustrated in FIG. 4A.

### Regional Mutations: Construction of Directed Mutant Gene Sets Containing Combinations of Substitution, Insertion, and Deletion Mutations.

To create a set of small insertions and deletions within the Ty3 GAG3 protein, the amino acid sequences of each desired GAG3 mutant protein are input into a system that identifies common and unique amino acid sequences among these different input sequences. These sequences are globally optimized by the sequence optimization system described in herein. Sequence-specific oligonucleotides are output and used to direct the self-assembly of genes for all of the input mutant protein sequences. An advantage of this assembly scheme is that it allows re-utilization of oligonucleotides for the common regions and building a set of mutant genes efficiently with a minimal number of oligonucleotides.

### Directed Shuffling: Directed Shuffling of DNA Sequences Among the Integrase (IN) Genes of the Yeast Ty3 Retrotransposon, the Moloney Marine Leukemia Virus (MMLV), and the Human Immunodeficiency Virus (HIV-1).

The nucleotide sequences coding for NTD1, NTD2, core, CTD1, and CTD2 protein domains from Ty3, MMLV, and HIV-1 IN genes are globally optimized as described herein. The result of this optimization is a total of 15 (five domains for each of three genes) unique, non-cross-hybridizing, self-assembling DNA sequences. To create all 75 possible combinations, primer sets are designed to direct the DNA shuffling events. For example, to join Ty3 NTD1 to MMLV NTD2, two primer sets are generated. The first primer set includes: (1) 5′ and 3′ end primers that amplify the Ty3 NTD1 sequence; and (2) a second primer set that amplifies the MLV NTD2 sequence. Complementary sequences of both primer sets are used to join these two amplification products, as described herein. This technique can be used to direct the combinatorial joining of the five domains of the TY3, MMLV, and HIV-1 IN gene sequences as illustrated in FIG. 13.

1. A composition, comprising:
a set of oligonucleotides configured to assemble into a group of non-overlapping polypeptide-encoding synthetic polynucleotides, wherein the oligonucleotides of the set have been mutually and globally thermodynamically optimized by computerized analysis, such that:
when Tmc represents the melting temperature of a correct hybridization between a given possible nucleotide internal sequence IS of length n and a fully complementary nucleotide sequence thereto ISC of length n, wherein n is selected to be at least 10; and
when Tmi represents the highest melting temperature of any possible incorrect hybridization between that same ISC and any other oligonucleotide of the set, or portion thereof;
there exists a temperature gap such that for each possible IS and corresponding ISC of the set, Tmc is higher than Tmi.

2. The composition of claim 1, wherein the group comprises at least 2 polynucleotides.

3. (canceled)

4. (canceled)

5. (canceled)

6. The composition of claim 1, wherein for all oligonucleotides of a selected subset of the entire set, the lowest Tmc of any fully complementary IS/ISC pair is higher than the highest Tmi associated with any ISC within the subset.

7. (canceled)

8. (canceled)

9. (canceled)

10. (canceled)

11. The composition of claim 1, wherein n is at least 15.

12. (canceled)

13. The composition of claim 1, wherein each polynucleotide in the group encodes at least a portion of a protein from the same protein family or superfamily.

14. (canceled)

15. A composition, comprising:
a set of oligonucleotides configured to assemble into a group of polynucleotides, each encoding a desired polypeptide; and
a primer having a first region that is fully complementary to a sequence S1 of a first polynucleotide of the polynucleotide group, wherein sequence S1 is of a minimum length of about 5 bases and having a second region that is fully complementary to a sequence S2 of a second polynucleotide of the polynucleotide group, wherein sequence S2 is of a minimum length of about 5 bases;
wherein codons of the oligonucleotides of the oligonucleotide set have been selected from among synonymous codons, and as a result, the melting temperature of the hybridization of the first region of the first primer to S1 is greater than the melting temperature of any incorrect hybridization of the first region to any other sequence of the set and the melting temperature of the hybridization of the second region of the second primer to S2 is greater than the melting temperature of any incorrect hybridization of the second region to any other sequence in the set.

16. The composition of claim 13, wherein S1 and S2 are of minimum length of about 10 bases.

17. (canceled)

18. A composition, comprising:
a set of oligonucleotides configured to assemble into a group of polynucleotides, each encoding a desired polypeptide; and
a primer pair, comprising a first and a second primer, wherein:
the first primer has a first region that is fully complementary to a sequence S1 of a first polynucleotide of the polynucleotide group, wherein sequence S1 is of a minimum length of about 5;
the second primer has a second region that is fully complementary to a sequence S2 of a second polynucleotide of the polynucleotide group, wherein sequence S2 is of a minimum length of about 5; and
the first primer has a third region that is identical to, or fully complementary to, a fourth region of the second primer, wherein the third region of the first primer includes none, part, or all of the first region of the first primer, and the fourth region of the second primer includes none, part, or all of the second region of the second primer; and
wherein codons of the oligonucleotides of the oligonucleotide set have been selected from among synonymous codons, and as a result, the melting temperature of the hybridization of the first region of the first primer to S1 is greater than the melting temperature of any incorrect hybridization of the first region to any other sequence of the set and the melting temperature of the hybridization of the second region of the second primer to S2 is greater than the melting temperature of any incorrect hybridization of the second region to any other sequence in the set.

19. The composition of claim 18, wherein S1 and S2 are of minimum length of about 10 bases.

20. (canceled)

21. The composition of claim 18, wherein the concentration of the primer pair is greater than the concentration of any oligonucleotide of any given sequence.

22. (canceled)

23. The composition of claim 18, wherein the third region of the first primer comprises a portion less than the entirety of the first region of the first primer and/or the fourth region of the second primer comprises a portion less than the entirety of the second region of the second primer.

24. (canceled)

25. (canceled)

26. A method for creating a chimeric polynucleotide from a set of oligonucleotides configured to assemble into a group of polynucleotides, comprising:
providing a set of oligonucleotides as set forth in claim 1; providing at least one primer, said primer having a first region uniquely complementary to a sequence of a first polynucleotide in the group and a second region uniquely complementary to a second polynucleotide of the group, and combining the primer with an oligonucleotide or polynucleotide comprising the first region of said first polynucleotide and an oligonucleotide or polynucleotide comprising the second region of said second polynucleotide; and
PCR amplifying to create a chimeric polynucleotide having some sequence from the first polynucleotide and some sequence from the second polynucleotide.

27. The method of claim 26, wherein said primer is between about 18 and about 25 bases in length.

28. The method of claim 26, wherein the concentration of the primer is greater than the concentration of any oligonucleotide of any given sequence.

29. (canceled)

30. A method for creating a plurality of chimeric polynucleotides, respectively encoding a plurality chimeric polypeptides, comprising:
providing a set of oligonucleotides as set forth in claim 1;
providing a plurality of different primers, each said primer having a first region uniquely complementary to a sequence of one of the polynucleotides of the group and a second region uniquely complementary to a sequence of another of the polynucleotides of the group, and, optionally, a third region not complementary to any polynucleotides of the group, wherein the plurality of primers differ from each other in at least the first region, the second region, or the third region;
contacting each of the primers with an oligonucleotide or polynucleotide complementary to the first or second regions to form primer-oligonucleotide or primer-polynucleotide hybridizations; and
PCR extending the hybridized primers to create chimeric polynucleotides.

31. The method of claim 30, wherein the group of polynucleotides contains at least 3 different polynucleotides and wherein each of the primers is contacted with assembled polynucleotides of the group.

32. The method of claim 30, wherein each of the primers is simultaneously contacted with assembled polynucleotides of the group.

33. (canceled)

34. (canceled)

35. (canceled)

36. A primer comprising a first region that is fully complementary to a sequence S1 of a first oligonucleotide of the oligonucleotide set of claim 1, wherein sequence S1 is of a minimum length of about 5 bases, said primer further comprising a second region that is fully complementary to a sequence S2 of a second oligonucleotide of the oligonucleotide set, wherein sequence S2 is of a minimum length of about 5 bases,
wherein the melting temperature of the hybridization of the first region to S1 is greater than the melting temperature of any incorrect hybridization of the first region to any other sequence of the set and the melting temperature of the hybridization of the second region to S2 is greater than the melting temperature of any incorrect hybridization of the second region to any other sequence in the set.

37. A primer pair, comprising a first and a second primer, wherein:
the first primer has a first region that is fully complementary to a sequence S1 of a first oligonucleotide of the oligonucleotide set of claim 1, wherein sequence S1 is of a minimum length of about 5;
the second primer has a second region that is fully complementary to a sequence S2 of a second oligonucleotide of the oligonucleotide set, wherein sequence S2 is of a minimum length of about 5; and
the first primer has a third region that is identical to, or fully complementary to, a fourth region of the second primer,
wherein the melting temperature of the hybridization of the first region of the first primer to S1 is greater than the melting temperature of any incorrect hybridization of the first region to any other sequence of the set and the melting temperature of the hybridization of the second region of the second primer to S2 is greater than the melting temperature of any incorrect hybridization of the second region to any other sequence in the set.
